(19) United States
(12) Patent Application Publication
(10) Pub. No.: US 2010/0104529 A1
(43) Pub. Date: Apr. 29, 2010

(54) TNF-alpha VACCINE FOR TREATING DISEASE CONDITIONS MEDIATED BY PATHOLOGICAL TNF-alpha

(76) Inventor: Robert L. England, Benicia, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/299,095

(22) PCT Filed: Apr. 17, 2007
(86) PCT No.: PCT/US07/09370

§ 371 Date: May 22, 2009

Related U.S. Application Data
(60) Provisional application No. 60/797,032, filed on May 1, 2006.

(51) Int. Cl. A61K 38/19; C07K 14/52; C07H 21/04; A61K 31/7088; C12N 15/63; C12N 5/10; A61P 25/00

(52) U.S. Cl. 424/85.2; 530/351; 536/23.5; 514/44 R; 435/320.1; 435/325

(57) ABSTRACT

The present invention provide TNFα-defensin/chemokine fusion polypeptides and polynucleotides and methods of treating, inhibiting or preventing disease conditions mediated by pathological TNFα, including chronic neuropathic pain, chronic inflammation, insulin resistance, diabetes, hyperlipidemia, hypertriglyceridemia, hypercholesterolemia and artherosclerosis, by administering a therapeutically effective amount of the TNFα-defensin/chemokine fusion polypeptides and polynucleotides.

### FIELD OF THE INVENTION

The present invention relates to defensin or chemokine and TNFα fusion proteins and their use in inducing an anti-TNFα immune response to counteract disease conditions mediated by pathological presence of TNFα, including but not limited to, chronic pain, chronic inflammatory conditions, diabetes, and cardiovascular disease (e.g., hypertriglyceridemia, hypercholesterolemia, artherosclerosis).

### BACKGROUND OF THE INVENTION

Tumor necrosis factor-alpha (TNFα) is a known source of pain generation in chronic neuropathic pain, including sciatica. See, Boos, et al., Spine (2002) 27:2631, Yoshida, et al., Spine (2005) 30:55, and Weiler, et al., Spine (2005) 30:44. TNFα has also been implicated in exacerbating or accelerating inflammatory, metabolic and cardiovascular disease processes, including those manifesting as rheumatoid arthritis, diabetes, hypertriglyceridemia, hypercholesterolemia and artherosclerosis. See, Popa, et al., J. Lipid Res., January 2007, PubMed ID: 17202130.

Treatments that neutralize the activity of TNFα, including anti-TNFα antibodies and soluble TNFα receptors, have been shown to be effective in treating chronic neuropathic pain, including back, neck and disk-related pain. See, Karppinen, et al., Spine (2003) 28:750, Tobinick, et al., Swiss Med Weekly (2003) 133:170 and Tobinick, et al., Curr Med Res Opin (2004) 20:1075. Treatments that neutralize the activity of TNFα also have been shown to be effective in improving insulin sensitivity in diabetic patients. See, for example, Ofei, et al., Diabetes (1996) 45:881, Bernstein, et al., Arch Intern Med (2006) 166:902-908, Yazdani-Biuki, et al., Eur J Clin Invest (2004) 34:641 and Huvers, et al, Ann Rheum Dis (2007) 66:558-9. Furthermore, statins and PPARγ agonists appear to improve lipid and glucose metabolic parameters in part through anti-inflammatory mechanisms. See, for example, Jain and Ridker, Nat Rev Drug Discov (2005) 4:977 and Lehrke, and Lazar, Cell (2005) 123:993.

Unfortunately, long-term injections of TNFα neutralizing moieties is extraordinarily expensive and carries potential risks, including infections and irritation from repeated injections and foreign antibody-induced autoimmune disorders, for example, systemic lupus erythematosus. See, Schaible, Can J Gastroenterol (2000) Suppl C:29C-32C, Moreland, et al., J Rheumatol (2001) 28:1238, Weycker, et al., Clin Ther (2005) 27:646.

Therefore, it would be desirable for an individual suffering from a disease condition in part generated or mediated by pathological TNFα production (e.g., chronic pain, diabetes, cardiovascular conditions including hyperlipidemia, hypercholesterolemia, artherosclerosis) to produce endogenous anti-TNFα neutralizing antibodies. Others have shown that immunizing mice with foreign peptide-TNFα fusion proteins to induce production of neutralizing antibodies against TNFα ameliorated symptoms of experimental cachexia, type II collagen induced arthritis and allergic inflammatory conditions. See, Dalum, et al., Nat Biotechnol (1999) 17:666, Zuany-Amorim, et al, Int Arch Allergy Immunol (2004) 133:154 and Chackerian, et al., J Clin Invest (2001) 108:415. However, it would be preferable that the tolerance-breaking antigen was not a foreign antigen.

It has been demonstrated that fusion proteins comprising a native defensin or native chemokine fused to a tumor antigen or viral antigen can elicit an immune response against the tumor antigen or viral antigen. See, U.S. Pat. No. 6,562,347 and U.S. Patent Publication No. 2005/0095257.

There currently exists a need for improved methods of neutralizing TNFα in disease conditions mediated at least in part by pathological TNFα, including chronic neuropathic pain conditions (e.g., chronic back, neck and disk-related pain), chronic inflammatory conditions, diabetes, and cardiovascular disease (e.g., hyperlipidemia, hypercholesterolemia, artherosclerosis). The present invention addresses this and other needs.

### BRIEF SUMMARY OF THE INVENTION

The present invention provides fusion compositions comprising a defensin or chemokine operably linked to a TNFα polypeptide and methods of treating TNFα-generated or TNFα-mediated pain by administering to a individual in need thereof a therapeutically effective amount of the present TNFα fusion compositions.

Accordingly, in a first aspect, the present invention provides fusion polypeptides comprising a TNFα polypeptide operably linked to a chemokine or a defensin. The TNFα polypeptide can comprise the N-terminal segment or the C-terminal segment of the polypeptide. In one embodiment, the chemokine or defensin comprise the N-terminal portion of the fusion polypeptide and the TNFα polypeptide comprises the C-terminal portion. In some embodiments, the chemokine or defensin and the TNFα polypeptide are from the same species (e.g., human, mouse, rat, etc.).

The TNFα polypeptide can be a full-length TNFα polypeptide or an immunogenic fragment of TNFα. Preferably, the immunogenic fragment elicits antibodies that neutralize the pain-generating activity of TNFα. In some embodiments, the immunogenic TNFα polypeptide fragments elicit antibodies that inhibit or prevent the binding of TNFα to a TNFα receptor, for example a type 1 TNFα receptor (TNFR1). In some embodiments, the immunogenic TNFα polypeptide fragments elicit antibodies that inhibit or prevent the binding of two or more TNFα monomers to each other. In some embodiments, the TNFα polypeptide comprises an amino acid sequence having at least 95% sequence identity to an amino acid sequence selected from the group consisting of SEQ ID NO:2, SEQ ID.NO:35, SEQ ID NO:38, SEQ ID NO:40, SEQ ID NO:43 and SEQ ID NO:46.

In some embodiments, the fusion polypeptides comprise a defensin. The defensin can be an alpha defensin or a beta defensin. In some embodiments, the defensin is an alpha defensin, for example, HNP-1, HNP-2 or HNP-3. In some embodiments, the alpha defensin comprises an amino acid sequence having at least 95% sequence identity to an amino acid sequence selected from the group consisting of SEQ ID NO:8, SEQ ID NO:10, SEQ ID NO:12, and SEQ ID NO:14. In some embodiment, the defensin is a beta defensin, for example, HBD1 or HBD2. In some embodiments, the beta defensin comprises an amino acid sequence having at least 95% sequence identity to an amino acid sequence selected from the group consisting of SEQ ID NO:16 and SEQ ID NO:18.

In some embodiments, the fusion polypeptides comprise a chemokine. The chemokine can be a CC chemokine or a CXC chemokine. The chemokine can be a full-length chemokine or a chemokine fragment that retains the chemotactic activity of a full-length chemokine. In some embodiments, the chemokine is selected from the group consisting of interferon-induced protein 10 (IP-10), monocyte chemotactic protein-1 (MCP-1), MCP-2, MCP-3, MCP-4, macrophage inflammatory protein 1 (MIP1), MIP2, MIP3, RANTES (CC chemokine ligand 5), macrophage-derived chemokine (MDC), stromal cell-derived factor 1 (SDF-1), and monokine induced by IFN-gamma (MIG). In some embodiments, the chemokine comprises an amino acid sequence having at least 95% sequence identity to an amino acid sequence selected from the group consisting of SEQ ID NO:24, SEQ ID NO:26 and SEQ ID NO:32.

In a further aspect, the invention provides compositions comprising a fusion polypeptide comprising a defensin or a chemokine operably fused to a TNFα polypeptide and a pharmaceutical carrier, for example, for administration to a subject as a vaccine.

In another aspect, the invention provides nucleic acids encoding a fusion polypeptide comprising a defensin or a chemokine operably linked to a TNFα polypeptide. The embodiments of the fusion polypeptides encoded by the nucleic acids are as described above and herein.

In some embodiments, the nucleic acids encode a fusion protein comprising a TNFα polypeptide encoded by a nucleic acid sequence having at least 95% sequence identity to a nucleotide sequence selected from the group consisting of SEQ ID NO:1, SEQ ID NO:49, SEQ ID NO:52, SEQ ID NO:54, SEQ ID NO:57, and SEQ ID NO:60.

In some embodiments, the nucleic acids encode a fusion protein comprising a defensin polypeptide encoded by a nucleic acid sequence having at least 95% sequence identity to a nucleotide sequence selected from the group consisting of SEQ ID NO:7, SEQ ID NO:9, SEQ ID NO:11, SEQ ID NO:13, SEQ ID NO:15 and SEQ ID NO:17.

In some embodiments, the nucleic acids encode a fusion protein comprising a chemokine polypeptide encoded by a nucleic acid sequence having at least 95% sequence identity to a nucleotide sequence selected from the group consisting of SEQ ID NO:23, SEQ ID NO:25, and SEQ ID NO:31.

In a further aspect, the invention provides compositions comprising a nucleic acid encoding a fusion polypeptide comprising a defensin or a chemokine operably linked to a TNFα polypeptide and a pharmaceutical carrier, for example, for administration to a subject as a vaccine.

In a further aspect, the invention comprises expression cassettes, vectors (plasmid or viral), and cells comprising a nucleic acid encoding a fusion polypeptide comprising a defensin or a chemokine operably linked to a TNFα polypeptide.

In another aspect, the invention provides a method for producing an immune response in a subject that is specifically directed against TNFα comprising administering to the subject a fusion polypeptide comprising a defensin or a chemokine operably linked to a TNFα polypeptide.

In a related aspect, the invention provides a method for producing an immune response in a subject that is specifically directed against TNFα comprising administering to the subject a nucleic acid encoding a fusion polypeptide comprising a defensin or a chemokine operably linked to a TNFα polypeptide.

In a further aspect, the invention provides a method of inhibiting or preventing chronic pain generated by or resulting from TNFα in a subject, comprising administering to the subject a fusion polypeptide comprising a defensin or a chemokine operably linked to a TNFα polypeptide.

In a related aspect, the invention provides a method of inhibiting or preventing chronic pain generated by or resulting from TNFα in a subject, comprising administering to the subject a nucleic acid encoding a fusion polypeptide comprising a defensin or a chemokine operably linked to a TNFα polypeptide.

In a further aspect, the invention provides a method of inhibiting or preventing a disease condition mediated by pathological TNFα in a subject comprising, administering to the subject a nucleic acid encoding a fusion polypeptide comprising a defensin or a chemokine operably linked to a TNFα polypeptide or a fusion polypeptide comprising a defensin or a chemokine operably linked to a TNFα polypeptide.

In carrying out the methods, the TNFα polypeptide and the defensin or chemokine are native to the subject being treated.

In some embodiments, the disease condition is selected from the group consisting of chronic inflammation, chronic neuropathic pain, diabetes and cardiovascular disease. In some embodiments, the chronic pain being treated is neuropathic pain. In some embodiments the chronic pain affects the back, neck, spine or shoulder of the individual.

The embodiments of the fusion polypeptides and nucleic acids used in the methods are as described above and herein.

### Definitions

The term “tumor necrosis factor-alpha” or “TNFα” polypeptide interchangeably refer to polypeptides and nucleic acid encoding polypeptides that structurally share at least about 90%, 95%, 97%, 98% or 99% amino acid or nucleic acid sequence identity with native TNFα sequences known in the art, for example, GenBank accession numbers NM_{—}000594 (human nucleotide), NP_{—}000585.2 (human protein), NM_{—}013693 (mouse nucleotide), NP_{—}038721.1 (mouse protein), NM_{—}012675 (rat nucleotide), and NP_{—}036807.1 (rat protein). The TNFα polypeptide can be a full-length protein, usually with the signal peptide removed, or can be immunogenic fragments. The TNFα polypeptides included in the fusion proteins of the invention specifically bind to antibodies raised against TNFα, but may or may not induce intracellular signaling through a TNFα receptor, for example TNFR1 or TNFR2.

The term “immunogenic fragment” refers to a polypeptide segment comprising an epitope that specifically binds to an anti TNFα antibody or a nucleic acid that encodes a polypeptide segment comprising an epitope that specifically binds to an anti TNFα antibody. An immunogenic polypeptide fragment is usually at least 10 amino acids in length, for example, about 15, 20, 25 or 30 amino acids in length, but is shorter than the full-length protein.

The term “epitope” or “antigenic determinant” refers to a site on a TNFα polypeptide to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding (i.e., conformationally determined) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996). Antibodies that recognize the same epitope can be identified in a simple immunoassay showing the ability of one antibody to block the binding of another antibody to a target antigen (e.g., a competitive ELISA or solid phase radioimmunoassay (SPRIA)). T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., J. Inf. Dis. 170, 1110-19 (1994)), by antigen-dependent killing (cytotoxic T lymphocyte assay, Tigges et al., J. Immunol. (1996) 156:3901-3910) or by cytokine secretion.

The terms “bind(s) specifically” or “specifically directed against” refers to the preferential association T-cell receptors and/or antibodies, in whole or part, with a TNFα polypeptide in comparison to other polypeptides. It is, of course, recognized that a certain degree of non-specific interaction may occur between an antibody or T-cell receptor and a non-TNFα polypeptide. Nevertheless, specific binding, may be distinguished as mediated through specific recognition of TNFα. Typically, specific binding or a specifically directed immune response results in a much stronger association between TNFα and an anti-TNFα antibody or T-cell receptor than between an anti-TNFα antibody or T-cell receptor and a non-TNFα polypeptide. Specific binding typically results in greater than about 10-fold and most preferably greater than 100-fold increase in amount of bound anti-TNFα antibody (per unit time) to a cell or tissue bearing TNFα as compared to a cell or tissue lacking a TNFα epitope. Specific binding between TNFα and an anti-TNFα antibody generally means an affinity of at least 10⁶ M⁻¹. Affinities greater than 10⁸ M⁻¹ are preferred. Specific binding can be determined using any assay for antibody binding known in the art, including Western Blot, ELISA, flow cytometry, immunohistochemistry. T-cells specifically directed against a TNFα epitope typically exhibit antigen-induced proliferation in response to a TNFα polypeptide that is greater than about 2-fold, and more preferably greater than about 5-fold or 10-fold than antigen-induced proliferation in response to a non-TNFα polypeptide. T cell proliferation assays are known in the art can be measured by ³H-thymidine incorporation.

The term “pathological TNFα ” refers to TNFα produced in vivo outside the context of a normal inflammatory or immune response. For example, TNFα continuously produced and detectably present in vivo in an individual for more than 3 weeks is considered pathological. TNFα chronically produced in the context of an inflammatory and/or immune response that does not resolve would be considered pathological. TNFα chronically produced and directly or indirectly causing tissue destruction or damage, hypertriglyceridemia, hypercholesterolemia, artherosclerosis, or insulin resistance is considered pathological.

The term “defensin” refers to an anti-microbial peptide with three to four intramolecular cysteine disulfide bonds which induces leukocyte migration in vitro, and/or enhances concavalin A-stimulated murine spleen cell proliferation and IFN-γ production (See, Tani, et al., Intl. Immunol (2000) 12(5): 691-700). A defensin can be either a naturally occurring defensin, or a synthetic defensin, such as a variant of a naturally occurring defensin, which may be chemically synthesized or produced by expressing a modified cDNA encoding a naturally occurring defensin. The defensin used will generally be from the species of the individual to be treated, for example, human, mouse, rat, dog, cat, horse, or any other mammal.

The defensins suitable for the fusion polypeptides of this invention can include, but are not limited to, alpha defensins and beta defensins. Typically, the defensins used in the fusion polypeptides of the invention are mature defensins, without a signal peptide or a propeptide sequence.

Alpha defensins include human neutrophil peptide-1 (HNP-1), human neutrophil peptide-2 (HNP-2), human neutrophil peptide-3 (HNP-3) (See, Tani, et al., supra and van Wetering, et al., J Allergy Clin Immunol (1999) 104(6):1131-1138), human neutrophil peptide-4 (HNP-4), human defensin-5 (HD-5), and human defensin-6 (HD-6). Human neutrophil defensins (HNPs 1-4) are small, cationic, and arginine rich peptides that lack enzymatic activity. The peptides contain six conserved cysteine residues that participate in 3 characteristic intramolecular disulfide bridges (See, van Wetering, supra). Cysteines of alpha-defensins are paired C₁-C₆, C₂-C₄ and C₃-C₅ (van Wetering, supra). Structurally, an alpha defensin generally comprises the following consensus sequence:

XC₁XC₂XXXXC₃XXXXXXXXXC₄XXXXXXXXXC₅C₆

Structurally, HNP-1, HNP-2 and HNP-3 comprise the following consensus sequence:

CYCRIPACIAGERRYGTCIYQGRLWAFCC

Beta defensins include β-defensin-1 (HBD1), human β-defensin-2 (HBD2) (van Wetering, supra, and Yang, et al., Science (1999) 286:525-528). Cysteine of beta-defensins connected C₁-C₅, C₂-C₄ and C₃-C₆ (van Wetering, supra). Structurally, a beta-defensin generally comprises the following consensus sequence:

XXXXC₁XXXXXXC₂XXXXC₃XXXXXXXXXC₄XXXXXXC₅C₆

The defensins in the fusion proteins of this invention can further include active fragments of defensins which retain the activity of the intact molecule, for example, chemotaxis and inhibition of chemotaxis. The production of active fragments of a defensin and identification of defensin fragments which retain the activity of the intact molecule are carried out according to protocols known in the art.

The term “chemokine” refers to a small secreted protein, induced by inflammatory stimuli. A chemokine can orchestrate a chemotactic response after binding to specific G-protein-coupled cell surface receptors (e.g. CXC chemokine receptor, CC chemokine receptor) on target cells (e.g., antigen presenting cells (APC), such as dendritic cells, monocytes, macrophages, keratinocytes and B cells), comprising the selective migration, diapedesis and activation of leukocytes which mediate the inflammatory response. As one example, the chemokine, interferon-induced protein 10 (IP-10) binds to the CXCR3 receptor, thus inducing chemotaxis of activated T cells, NK cells, etc., which express this receptor. As another example, the chemokine monocyte chemotactic protein-3 (MCP-3) acts via binding to the CCR1, CCR2 and CCR3 chemokine receptors on antigen presenting cells (APC) including dendritic cells, eosinophils, basophils, monocytes and activated T cells. Thus, MCP-3 selectively targets and induces chemotaxis of these cell types.

Chemokine polypeptides suitable in the fusion proteins of the invention can include, but are not limited to, interferon-induced protein 10 (IP-10), monocyte chemotactic protein-1 (MCP-1), MCP-2, MCP-3, MCP-4, macrophage inflammatory protein 1 (MIP1), MIP2, MIP3, RANTES (CC chemokine ligand 5), macrophage-derived chemokine (MDC), stromal cell-derived factor 1 (SDF-1), monokine induced by IFN-gamma (MIG), as well as any other chemokine now known or later identified.

The chemokines in the fusion proteins can also include active fragments of chemokines which retain the chemotactic activity of the intact molecule. A chemokine generally comprises two structural portions: the amino terminal portion and the carboxy terminal portion. The amino terminal portion is responsible for chemokine receptor binding and the carboxy terminal end binds to heparin and heparan sulfate, for example, in the extracellular matrix and on the surface of endothelial cells. For example, for both CC and CXC chemokines, the N-terminal region is the critical region of the molecule for biological activity and leukocyte selectivity. In particular, the N-terminal ELR motif-containing CXC chemokines are chemotactic for neutrophils, whereas those not containing the motif act on lymphocytes. IP-10 and MIG, for example, do not contain the ELR motif and are known to attract activated T cells (Clark-Lewis, et al., J Biol Chem (1991) 266:128-134). In a further example, addition of a single amino acid residue to the amino terminus of MCP-1 decreases its biological activity up to 1000 fold and deletion of a single amino acid for that region converts the chemokine from an activator of basophils to an eosinophil chemoattractant (Weber, et al., Exp Med (183:681-685).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 90% identity, preferably 90%, 91%, 92%, 93%, 94%, 95%, 96%, 97%, 98%, 99%, or higher identity over a specified region (e.g., any one of SEQ ID NOs:1-62), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or can be applied to, the compliment of a test sequence. The definition also includes sequences that have deletions and/or additions, as well as those that have substitutions. As described below, the preferred algorithms can account for gaps and the like. Preferably, identity exists over a region that is at least about 25, 50, 75 or 100 amino acids or nucleotides in length or over the full-length of am amino acid or nucleic acid sequences.

For sequence comparison, typically one sequence acts as a reference sequence, to which test sequences are compared (for example, a full-length TNFα amino acid or nucleic acid sequence or fragment thereof). When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A preferred example of algorithm that is suitable for determining percent sequence identity and sequence similarity are the BLAST algorithms, which are described in Altschul et al., Nuc. Acids Res. 25:3389-3402 (1977) and Altschul et al., J. Mol. Biol. 215:403-410 (1990), respectively. BLAST software is publicly available through the National Center for Biotechnology Information on the worldwide web at ncbi.nlm.nih.gov/. Both default parameters or other non-default parameters can be used. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” as used herein applies to amino acid sequences. One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins (1984)).

The terms “mammal” or “mammalian” refer to any animal within the taxonomic classification mammalia. A mammal can refer to a human or a non-human primate. A mammal can refer to a domestic animal, including for example, canine, feline, rodentia, including lagomorpha, murine, rattus, Cricetinae (hamsters), etc. A mammal can refer to an agricultural animal, including for example, bovine, ovine, porcine, equine, etc.

The term “operably linked” refers to a functional linkage between a first nucleic acid sequence and a second nucleic acid sequence, such that the first and second nucleic acid sequences are transcribed into a single nucleic acid sequence. Operably linked nucleic acid sequences need not be physically adjacent to each other. The term “operably linked” also refers to a functional linkage between a nucleic acid expression control sequence (such as a promoter, or array of transcription factor binding sites) and a transcribable nucleic acid sequence, wherein the expression control sequence directs transcription of the nucleic acid corresponding to the transcribable sequence. “Operably linked” also refers to the functional linkage of a first polypeptide to a second polypeptide in the formation of a fusion protein. Again, the fused polypeptides need not be physically adjacent to each other, but can be joined through a linker, for example a flexible Ser-Gly linker.

As used herein, the term “chronic disease condition” refers to refers to a pathological condition suffered by an individual for at least 3 weeks, oftentimes 3 or more months, without resolution. Chronic disease conditions often escalate over time, even with treatment. Exemplified chronic disease conditions secondary to the continuous pathological production TNFα include chronic pain conditions, chronic inflammation, hypertriglyceridemia, hypercholesterolemia, artherosclerosis, insulin resistance and diabetes.

As used herein, the term “chronic pain” refers to neuropathic pain, for example, due to injury to a peripheral or central nervous pathway.

The term “patient”, “individual” or “subject” refers to any mammal, for example, a human, non-human primate, a domestic mammal (e.g., a canine or feline), an agricultural mammal (e.g., equine, bovine, ovine, porcine), or a laboratory mammal or rodent (e.g., rat, mouse, hamster, rabbit).

### DETAILED DESCRIPTION

1. Introduction

The present invention provides fusion polypeptides and nucleic acids encoding fusion polypeptides comprising a TNFα polypeptide and a defensin polypeptide or chemokine polypeptide. The invention further provides methods of treating (i.e., ameliorating or preventing) chronic disease conditions that result directly or indirectly from continuous, chronic or unresolving production of TNFα, including for example, chronic pain conditions, chronic inflammation, hypertriglyceridemia, hypercholesterolemia, artherosclerosis, insulin resistance and diabetes. In some embodiments, the invention provides methods of treating chronic neuropathic pain generated by TNFα. Generally, the methods are performed by administering a fusion polypeptide or a nucleic acid encoding a fusion polypeptide comprising a TNFα polypeptide and a defensin or chemokine. Both polypeptides of the fusion protein, the TNFα polypeptide and the defensin polypeptide or chemokine polypeptide, are native to the species being treated for the chronic disease condition secondary to pathological production of TNFα. Without being bound to any theory, immunization with fusion polypeptides comprising a TNFα polypeptide and a defensin polypeptide or chemokine polypeptide facilitates presentation on antigen presenting cells (APCs), particularly dendritic cells, of immunogenic TNFα epitopes. Immunization or vaccination of an individual with the fusion polypeptides of the invention provides a method for treating chronic disease conditions caused by pathological TNFα without exposing the individual to a foreign antigen.

Substantial evidence supports the pathological role contributed by chronic TNFα production in the progression and exacerbation of chronic disease conditions, including chronic inflammatory conditions, hypertriglyceridemia, hypercholesterolemia, artherosclerosis, insulin resistance and diabetes. See, Popa, et al., J. Lipid Res., Jan. 2007, PubMed ID: 17202130. With respect to the role of pathological TNFα in the progression and/or exacerbation of chronic neuropathic pain, chronic neuropathic pain presents a distinct pathology from chronic inflammatory and (auto)immune-mediated pathologic conditions, for example, rheumatoid arthritis. Chronic neuropathic pain can manifest without inflammation or tissue pathology (see, Chapter 18 of Guides to the Evaluation of Permanent Impairment, Cocchiarella and Andersson, eds., Fifth Edition, 2001, American Medical Association Press and Chapter 8 of Low Back Pain: A Symptom-Based Approach to Diagnosis and Treatment, Rucker, et al., eds., 2001, Butterworth-Heinemann). For example, intervertebral disks can release neuropeptides, cytokines (e.g., TNFα), or other substances that can stimulate free nerve endings (see, Managing Low Back Pain, Kirkaldy-Willis and Bernard, eds., Fourth Edition, 1999, Churchill Livingstone; Igarashi, et al., Spine (2000) 200F;25(23):2975-80; Seguin, et al., Spine (2005) 30:1940-8; and Mulleman, et al., Joint Bone Spine (2006) 73:151-8). Neuropathic pain has been correlated with the presence of TNFα and TNFα signaling through type 1 TNFα receptors (TNFR1) (see, Sommer, et al., Exp Neurol (1998) 151:138; Sommer, Schmerz (1999) 13:315-23; Empl, et al., Neurology (2001) 56:1371-77 and Moalem and Tracey, Brain Research Rev (2005) December 30; PMID: 16388853) independent of inflammation.

2. Compositions

a. Fusion Polypeptides and Nucleic Acid Encoding Fusion Polypeptides

Generally, the fusion polypeptides comprise TNFα and defensin or chemokine amino acid or nucleic acid sequences based on the native sequences from the species intended to be treated. For example, for treating a human suffering from chronic pain caused at least in part by TNFα, a fusion protein comprising a human TNFα polypeptide and a human defensin or a human chemokine is administered.

i. TNF-Alpha

The TNFα amino acid and nucleic acid sequences in the fusion polypeptides of the invention are based on native mammalian TNFα sequences as a template. Nucleic acid and amino acid sequences encoding native mammalian TNFα can be readily found in publicly available databases including nucleotide, protein and scientific databases available on the worldwide web through the National Center for Biotechnology Information at ncbi.nlm.nih.gov. Native TNFα nucleic acid sequences can be conveniently cloned from monocytes/macrophages, NK cells and/or T cells (see, Janeway, et al., Immunobiology, 5^{th} edition, 2001). Protocols for isolation and stimulation of desired immune cell populations are well known in the art. See, for example, Current Protocols in Immunology, Coligan, et al., eds., 1991-2006, John Wiley & Sons. General protocols for cloning gene sequences can be found, for example, in Sambrook and Russell, Molecular Cloning: A Laboratory Manual, 2001, Cold Spring Harbor Laboratory Press and Ausubel, et al., Current Protocols in Molecular Biology, 1987-2007, John Wiley & Sons.

The TNFα polypeptide can be at the N-terminus or the C-terminus of the fusion polypeptide. In some embodiments, the TNFα polypeptide comprises the C-terminus of the fusion polypeptide. In some embodiments, the fusion polypeptide will comprise a full-length TNFα polypeptide. In some embodiments, a mature TNFα polypeptide is used, although in some instances, it may be desirable to include a signal peptide, either a native TNFα polypeptide signal peptide or a signal peptide from a heterologous protein.

In some embodiments, the fusion polypeptide comprises an immunogenic fragment of a TNFα polypeptide. The immunogenic fragment can comprise a contiguous epitope or a discontinous, conformationally determined epitope. Suitable immunogenic fragments will elicit antibodies and/or T-cell responses that inhibit or prevent the cytolytic or pain-generating activity of a full-length TNFα polypeptide. For example, suitable TNFα immunogenic fragments will elicit antibodies that interfere with trimer formation of full-length TNFα polypeptide monomers and/or inhibit the binding of a full-length TNFα polypeptide to its cognate receptor. Exemplified immunogenic fragments of TNFα include amino acids 1-23 (SEQ ID NOs: 35-37 and 49-51), 37-55 (SEQ ID NOs:38-39 and 52-53), 95-116 (SEQ ID NOs:43-45 and 57-59), 117-136 (SEQ ID NOs:40-42 and 54-56), and 137-157 (SEQ ID NOs:46-48 and 60-62).

Numerous methods for determining immunogenic B-cell and/or T-cell epitopes of a polypeptide are known in the art. For identification of continuous epitopes, the methods can employ synthesizing overlapping peptides with sequences corresponding to the full-length of a polypeptide (e.g., TNF-alpha) to be mapped, e.g., amino acids 1-20, aa 10-30, aa 20-40, etc. Binding of the peptide sequences to antibodies or T-cell receptors directed against the polypeptide of interest (e.g. TNF-alpha) can be measured using well known methods, including ELISPOT, mass spectrometry, solid phase radioimmunoassay (SPRIA), ELISA, PEPSCAN, and T-cell proliferation assays. Methods for determining immunogenic epitopes are described, for example, in Reineke, et al., Curr Top Microbiol Immunol (1999) 243:23-36; Mahler, et al., Clin Immunol (2003) 107:65; Anthony and Lehmann, Methods (2003) 29:260; Parker and Tomer, Methods Mol Biol (2000) 146:185; DeLisser, Methods Mol Biol (1999) 96:11; Van de Water, et al., Clin Immunol Immunopathol (1997) 85:229; Carter, Methods Mol Biol (1994) 36:207; and Pettersson, Mol Biol Rep (1992) 16:149.

ii. Defensins

The fusion polypeptides can comprise a defensin, including without limitation, an alpha defensin or a beta defensin. The defensin amino acid and nucleic acid sequences in the fusion polypeptides of the invention are based on native mammalian defensin sequences as a template. Native defensins can be conveniently cloned from numerous tissues, including neutrophils, intestinal Paneth cells, or epithelial cells such as those of the skin, kidney, and trachea-bronchial lining using techniques known in the art. The fusion polypeptides preferably will comprise a mature defensin, lacking a signal peptide and/or propeptide sequence. Defensin amino acid and nucleic acid sequences from several mammalian species are known in the art, and can be found on the worldwide web at ncbi.nlm.nih.gov. Primers for cloning defensins are disclosed, for example, in U.S. Patent Publication 2005/0095257, hereby incorporated herein by reference in its entirety for all purposes.

The defensin can comprise the N-terminus or the C-terminus of the fusion polypeptide. In some embodiments, the defensin comprises the N-terminus of the fusion polypeptide.

iii. Chemokines

The fusion polypeptides can comprise a chemokine, including a CC chemokine or a a CXC chemokine. The chemokine amino acid and nucleic acid sequences in the fusion polypeptides of the invention are based on native mammalian chemikine sequences as a template. Chemokines suitable for inclusion in the present fusion proteins include, without limitation, interferon-induced protein 10 (IP-10), monocyte chemotactic protein-1 (MCP-1), MCP-2, MCP-3, MCP-4, macrophage inflammatory protein 1 (MIP1), MIP2, MIP3; RANTES (CC chemokine ligand 5), macrophage-derived chemokine (MDC), stromal cell-derived factor 1 (SDF-1), monokine induced by IFN-gamma (MIG). Numerous cell types can secrete chemokines, including fibroblasts, endothelial cells, epithelial cells, monocytes, macrophages, T cells, B cells, PMNs. Chemokine secretion can be induced by proinflammatory cytokines, including interferon-gamma, interleukin 4, products of Th1 and Th2 lymphocytes, interleukin-1, tumor necrosis factor-alpha and bacterial products such as lipopolysaccharide, as well as viral infection (Baggioni, et al., Adv. Immunol (1994) 55:97-179 and Garcia-Zepeda, et al., J. Immunol. (1996) 157:5613-5626). In some embodiments, the fusion polypeptides comprise a mature chemokine, lacking a native signal peptide and/or propeptide sequence. Chemokine amino acid and nucleic acid sequences from several mammalian species are known in the art, and can be found at ncbi.nlm.nih.gov. Primers for cloning chemokines are disclosed, for example, in U.S. Pat. No. 6,562,347 and in U.S. Patent Publication 2005/0095257, hereby incorporated herein by reference in their entirety for all purposes. In some embodiments, the fusion polypeptides comprise a viral chemokine (see, U.S. Patent Publication 2004/0110165).

The chemokine can comprise the N-terminus or the C-terminus of the fusion polypeptide. In some embodiments, the chemokine comprises the N-terminus of the fusion polypeptide.

iv. Construction of Fusion Proteins

The TNFα fusion polypeptides of the invention can be synthetically constructed, for example by a commercial service that specializes in the synthetic construction of custom amino acid sequences. An exemplified commercial provider of synthetic custom amino acid sequences is CSS, Chemical Synthesis Services, East Lothian, Scotland. CSS also specializes in the synthesis of defensins and chemokines.

Nucleotide sequences can also be constructed that encode the TNFα fusion polypeptides of the invention. One technique for joining a TNFα nucleotide sequence with a defensin or a chemokine nucleotide sequence includes splice-overlap PCR. The TNFα nucleotide sequence and a defensin or chemokine nucleotide sequences can be amplified and joined in a single reaction tube as a single sequence, using a “splice-overlap” PCR technique. Primers are designed such that the reverse primer for amplifying the N-terminal sequence portion and the forward primer for amplifying the C-terminal sequence portion produce overhangs that anneal to each other (see, for example, Warrens, et al., Gene (1997) 186:29-35). Alternatively, the TNFα nucleotide sequence and the defensin or chemokine sequence can be amplified as individual sequences and cloned into a cloning vector or an expression vector in a tripartite ligation reaction. Polynucleotides encoding the TNFα-defensin or chemokine fusion proteins of the invention also can be custom synthesized by a commercial provider, for example, Celtek Genes of Nashville, Tenn.; DNA 2.0 of Menlo Park, Calif.; and Integrated DNA Technologies, Coralville, Iowa.

The TNFα portion and the defensin or chemokine portion of the fusion polypeptide can be directly abutting or can be joined through a linker, for example, a flexible Gly₄Ser linker comprising 1-5 Gly₄Ser units. Nucleotide sequences encoding the TNFα fusion polypeptides can comprise a sequence coding for an intervening linker that operably links the TNFα coding portion to the defensin or chemokine coding portion.

Once a TNFα-defensin/chemokine fusion nucleic acid sequence has been constructed, it can be cloned into a cloning vector, for example a TA-cloning® vector (Invitrogen, Carlsbad, Calif.) before subjecting to further manipulations for insertion into one or more expression vectors. Manipulations of TNFα-defensin/chemokine fusion nucleic acid sequences, including recombinant modifications and purification, can be carried out using procedures well known in the art. Such procedures have been published, for example, in Sambrook and Russell, Molecular Cloning: A Laboratory Manual, 2001, Cold Spring Harbor Laboratory Press and Current Protocols in Molecular Biology, Ausubel, et al., eds., 1987-2007, John Wiley & Sons.

v. Expression Cassettes

The TNFα-defensin/chemokine fusion proteins can be recombinantly expressed from an expression vector containing a TNFα-defensin/chemokine fusion coding sequence. The expression vectors of the invention have an expression cassette that will express a TNFα-defensin/chemokine fusion polypeptide in a host cell, for example, bacteria, yeast, insect or mammalian. Within each expression cassette, sequences encoding a TNFα-defensin/chemokine fusion will be operably linked to expression regulating sequences. “Operably linked” sequences include both expression control sequences that are contiguous with the nucleic acid of interest and expression control sequences that act in trans or at a distance to control the gene of interest. Expression control sequences include appropriate transcription initiation, termination, promoter and enhancer sequences; efficient RNA processing signals such as splicing and polyadenylation signals; sequences that enhance translation efficiency (e.g., Kozak consensus sequence); sequences that enhance protein stability; and when desired, sequences that enhance protein secretion.

The expression vector can optionally also have a third independent expression vector for expressing a selectable marker. Selectable markers are well known in the art, and can include, for example, proteins that confer resistance to an antibiotics, fluorescent proteins, antibody epitopes, etc. Exemplified markers that confer antibiotic resistance include sequences encoding β-lactamases (against β-lactams including penicillin, ampicillin, carbenicillin), or sequences encoding resistance to tetracylines, aminoglycosides (e.g., kanamycin, neomycin), etc. Exemplified fluorescent proteins include green fluorescent protein, yellow fluorescent protein and red fluorescent protein.

vi. Vectors

In order to express a desired polypeptide, the nucleotide sequences encoding the TNFα-defensin/chemokine fusion polypeptide, or functional equivalents, can be inserted into appropriate expression vector, i.e., a vector which contains the necessary elements for the transcription and translation of the inserted coding sequence. Methods which are well known to those skilled in the art can be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook and Russell, supra and Ausubel, et al., supra.

A variety of expression vector/host systems can be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal (e.g., mammalian) cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors can be selected depending upon the use intended for the expressed polypeptide. For example, when large quantities are needed, for example for the induction of antibodies, vectors which direct high level expression of fusion proteins that are readily purified can be used. Such vectors include, but are not limited to, multifunctional E. coli cloning and expression vectors encoding purification tags that are expressed in frame with the TNFα-defensin/chemokine fusion sequences, including HisTag (6 or more sequential histidines), FLAG, Glutathione S-transferase (GST). Numerous expression vectors for use in bacterial host cells are commercially available, for example, from Novagen/EMD Biosciences, San Diego, Calif.; Stratagene, La Jolla, Calif.; Invitrogen, Carlsbad, Calif.; and Promega, Madison, Wis. In general, such fusion proteins are soluble and can easily be purified from lysed cells by adsorption to, for example, nickel agarose or glutathione-agarose beads followed by elution in the presence of free imidazole or glutathione. Proteins made in such systems can be designed to include heparin, thrombin, or factor Xa protease cleavage sites so that the cloned polypeptide of interest can be released from the GST moiety at will.

In yeast, including Saccharomyces or Pichia, a number of vectors containing constitutive or inducible promoters such as alpha factor, alcohol oxidase, and PGH can be used. For reviews, see Ausubel et al. (supra). Yeast expression vectors are commercially available from, for example, Invitrogen, Stratagene and Dualsystems Biotech AG, Zürich, Switzerland.

In cases where plant expression vectors are used, the expression of sequences encoding polypeptides May be driven by any of a number of promoters. For example, viral promoters such as the 35S and 19S promoters of CaMV may be used alone or in combination with the omega leader sequence from TMV (Takamatsu, EMBO J. 6:307 311 (1987)). Alternatively, plant promoters such as the small subunit of RUBISCO or heat shock promoters may be used (Coruzzi et al., EMBO J. 3:1671 1680 (1984); Broglie et al., Science 224:838 843 (1984); and Winter et al., Results Probl. Cell Differ. 17:85 105 (1991)). These constructs can be introduced into plant cells by direct DNA transformation or pathogen-mediated transfection. Such techniques are described in a number of generally available reviews (see, e.g., Hobbs in McGraw Hill Yearbook of Science and Technology pp. 191-196 (1992)). Suitable vectors can be found, for example, on the worldwide web through arabidopsis.org.

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the fusion polypeptide can be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses can then be used to infect, for example, S. frugiperda cells or Trichoplusia larvae in which the polypeptide of interest may be expressed (Engelhard et al., Proc. Natl. Acad. Sci. U.S.A. 91:3224 3227 (1994)). Insect cell expression vectors are commercially available from, for example, Invitrogen, Novagen, and BDBiosciences, San Jose, Calif.

In mammalian host cells, a number of viral-based expression systems are generally available. For example, in cases where an adenovirus is used as an expression vector, sequences encoding a polypeptide of interest can be ligated into an adenovirus transcription/translation complex consisting of the late promoter and tripartiteleader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655 3659 (1984)). In addition, transcription enhancers, such as the Rous sarcoma virus (RSV) enhancer, may be used to increase expression in mammalian host cells.

Other expression systems are available for expression of the TNFα-defensin/chemokine fusion polypeptides in a mammalian cell. The promoter or promoters can be viral, oncoviral or native mammalian, constitutive or inducible, or can preferentially regulate transcription of the fusion polypeptides in a particular tissue type or cell type (e.g., “tissue-specific”). Vectors for expression of proteins in mammalian host systems are commercially available from numerous sources, including Invitrogen, Novagen, Promega, Stratagene, and New England Biolabs, Ipswich, Mass.

In some embodiments the fusion polypeptides can be expressed from a constitutive promoter. A “constitutive” promoter is a promoter that is active under most environmental and developmental conditions. Exemplified constitutive promoters in mammalian cells include oncoviral promoters (e.g., simian cytomegalovirus (CMV), human CMV, simian virus 40 (SV40), rous sarcoma virus (RSV)), promoters for immunoglobulin elements (e.g., IgH), promoters for “housekeeping” genes (e.g., β-actin, dihydrofolate reductase).

In another embodiment, inducible promoters may be desired. An “inducible” promoter is a promoter that is active under environmental or developmental regulation. Inducible promoters are those which are regulated by exogenously supplied compounds, including without limitation, a zinc-inducible metallothionine (MT) promoter; an isopropyl thiogalactose (IPTG)-inducible promoter, a dexamethasone (Dex)-inducible mouse mammary tumor virus (MMTV) promoter; a tetracycline-repressible system (Gossen et al, Proc. Natl. Acad. Sci. USA, 89: 5547-5551 (1992)); the tetracycline-inducible system (Gossen et al., Science, 268: 1766-1769 (1995); see also Harvey et al., Curr. Opin. Chem. Biol., 2: 512-518 (1998)); the RU486-inducible system (Wang et al., Nat. Biotech., 15: 239-243 (1997) and Wang et al., Gene Ther., 4: 432-441 (1997)); and the rapamycin-inducible system (Magari et al. J. Clin. Invest., 100: 2865-2872 (1997)). Other types of inducible promoters which can be useful in this context are those which are regulated by a specific physiological state, e.g., temperature, acute phase, or in replicating cells only.

In another embodiment, the native promoter for a mammalian TNFα, a defensin, or a chemokine can be used. The native promoter may be preferred when it is desired that expression of the TNFα-defensin/chemokine fusion sequences should mimic the native expression of one polypeptide member of the fusion protein, TNFα, defensin, or chemokine. The native promoter can be used when expression of the TNFα-defensin/chemokine fusion sequences is desired to be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic expression of one polypeptide member of the fusion protein.

In another embodiment, the TNFα-defensinlchemokine fusion sequences can be operably linked to a tissue-specific promoter. For instance, if expression in lymphocytes or monocytes is desired, a promoter preferentially active in lymphocytes or monocytes, respectively, should be used. Examples of promoters that are tissue-specific are known for numerous tissues, including liver (albumin, Miyatake et al. J. Virol., 71: 5124-32 (1997); hepatitis B virus core promoter, Sandig et al., Gene Ther., 3: 1002-9 (1996); alpha-fetoprotein (AFP), Arbuthnot et al., Hum. Gene Ther. 7: 1503-14 (1996)), bone (osteocalcin, Stein et al., Mol. Biol. Rep., 24: 185-96 (1997); bone sialoprotein, Chen et al., J. Bone Miner. Res., 11: 654-64 (1996)), lymphocytes (CD2, Hansal et al., J. Immunol., 161: 1063-8 (1998); immunoglobulin heavy chain; T cell receptor a chain), neuronal (neuron-specific enolase (NSE) promoter, Andersen et al. Cell. Mol. Neurobiol., 13: 503-15 (1993); neurofilament light-chain gene, Piccioli et al., Proc. Natl. Acad. Sci. USA, 88: 5611-5 (1991); the neuron-specific vgf gene, Piccioli et al., Neuron, 15: 373-84 (1995)); among others.

For application to expression systems generally, specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125 162 (1994)).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1 to 2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 11:223 32 (1977)) and adenine phosphoribosyltransferase (Lowy et al., Cell 22:817 23 (1990)) genes which can be employed in tk⁻ or aprt⁻ cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567 70 (1980)); npt, which confers resistance to the aminoglycosides, neomycin and G-418 (Colbere-Garapin et al., J. Mol. Biol. 150:1 14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047 51 (1988)). Visible markers, including anthocyanins, β-glucuronidase and its substrate GUS, and luciferase and its substrate luciferin, and fluorescent proteins (e.g., red, green, yellow) also find use.

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells which contain and express a desired polynucleotide sequence may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton et al., Serological Methods, a Laboratory Manual (1990) and Maddox et al., J. Exp. Med. 158:1211 1216 (1983).

Host cells transformed with a polynucleotide sequence of interest can be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention can be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide can be used to facilitate purification. One such expression vector provides for expression of a fusion . protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath et al., Prot. Exp. Purif. 3:263 281 (1992), while the enterokinase cleavage site provides a means for purifying the desired polypeptide from the fusion protein. A discussion of vectors which contain fusion proteins is provided in Kroll et al., DNA Cell Biol. 12:441 453 (1993)).

vii. Cells

The invention further provides for host cells comprising a TNFα-fusion nucleotide or polypeptide sequence, including mammalian, insect, yeast, and bacterial host cells. Mammalian host cells can be in vivo in a host or in vitro. For example, expression vectors containing the TNFα-defensin/chemokine fusion nucleic acid sequences can be transfected into cultured mammalian host cells in vitro, or delivered to a mammalian host cell in a mammalian host in vivo.

Exemplary host cells that can be used to express the TNFα-defensin/chemokine fusion sequences include mammalian primary cells and established mammalian cell lines, including COS, CHO, HeLa, NIH3T3, HEK 293-T, RD and PC12 cells. Mammalian host cells for expression of the TNFα-defensin/chemokine fusion sequences are commercially available from, for example, the American Type Tissue Collection (ATCC), Manassas, Va. Protocols for in vitro culture of mammalian cells are also well known in the art. See, for example, Handbook of Industrial Cell Culture: Mammalian, Microbial, and Plant Cells, Vinci, et al., eds., 2003, Humana Press; and Mammalian Cell Culture: Essential Techniques, Doyle and Griffiths, eds., 1997, John Wiley & Sons.

Protocols for transfecting mammalian host cells in vitro and expressing recombinant nucleic acid sequences are well known in the art. See, for example, Sambrook and Russell, and Ausubel, et al, supra; Gene Delivery to Mammalian Cells: Nonviral Gene Transfer Techniques, Methods in Molecular Biology series, Heiser, ed., 2003, Humana Press; and Makrides, Gene Transfer and Expression in Mammalian Cells, New Comprehensive Biochemistry series, 2003, Elsevier Science. Mammalian host cells modified to express the TNFα-defensin/chemokine fusion sequences can be transiently or stably transfected with a recombinant vector. The TNFα-defensin/chemokine fusion sequences can remain epigenetic or become chromasomally integrated.

b. Pharmaceutical Compositions

The invention contemplates compositions comprising TNFα-defensin/chemokine fusion polypeptide and polynucleotide sequences in a physiologically acceptable carrier. While any suitable carrier known to those of ordinary skill in the art may be employed in the pharmaceutical compositions of this invention, the type of carrier will vary depending on the mode of administration. For parenteral administration, including intradermal, subcutaneous or intramuscular injection, the carrier preferably comprises water, saline, and optionally an alcohol, a surfactant, a fat, a polymer, a wax, one or more stabilizing amino acids or a buffer. General formulation technologies are known to those of skill in the art (see, for example, Remington: The Science and Practice of Pharmacy (21st edition), University of the Sciences in Philadelphia (USIP), 2005, Lippincott Williams & Wilkins; Injectable Dispersed Systems: Formulation, Processing And Performance, Burgess, ed., 2005, CRC Press; and Pharmaceutical Formulation Development of Peptides and Proteins, Frkjr et al., eds., 2000, Taylor & Francis).

The pharmaceutical compositions can further comprise one or more adjuvants. A “suitable adjuvant” refers to a substance that enhances an immune response in a subject without deleterious effect on the subject. Suitable adjuvants include, but are not limited to, an immunostimulatory cytokine, SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Other suitable adjuvants are well known in the art and include QS-21, Freund's adjuvant (complete and incomplete), alum, aluminum phosphate, aluminum hydroxide, N-acetyl-muramyl-L-threonyl-D-isoglutamine (thr-MDP), N-acetyl-normuramyl-L-alanyl-D-isoglutamine (CUP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion. The adjuvant can be administered before the administration of the fusion protein or nucleic acid encoding the fusion protein, concurrent with the administration of the fusion protein or nucleic acid or up to five days after the administration of the fusion polypeptide or nucleic acid to a subject. QS-21, similarly to alum, complete Freund's adjuvant, SAF, etc., can be administered within hours of administration of the fusion protein.

3. Methods

The TNFα-defensin/chemokine fusion polypeptides and nucleotides encoding the TNFα-defensin/chemokine fusion polypeptides find use in treating, ameliorating, inhibiting or preventing chronic disease conditions secondary to the continuous pathological production of TNFα, including chronic pain conditions, chronic inflammatory conditions, insulin resistance, diabetes, hypertriglyceridemia, hypercholesterolemia and artherosclerosis. In some embodiments, the present methods are used to inhibit or prevent chronic pain resulting from or mediated by TNFα. In general, the chronic pain is neuropathic pain, for example, due to injury to a central or a peripheral nervous pathway. Usually, pain is considered chronic if it lasts for 12 weeks or longer. In some embodiments, the chronic pain can be due to a peripheral or cervical nerve root injury or nerve root compression. In some embodiments, the chronic pain is experienced by the subject in the neck, shoulder, arms, hands, legs, feet, spine, upper back (e.g., cervical, thoracic), lower back (e.g., lumbar, sacral), or pelvis. The chronic pain can result from physical trauma, for example, to the neck, shoulders or spine, or as a result of a degenerative spinal disease. In some embodiments, the methods are applied to the treatment of cervical disk disease, lumbar disk disease, intervertebral disk degeneration, or herniated disk. See, Part II, Chapters 11 and 15 of Harrison's Principles of Internal Medicine, Kasper, et al., eds., 16^{th} edition, 2005, McGraw-Hill. In some embodiments, the methods are applied to the treatment of chronic neuropathic pain that manifests without evidence of inflammation or tissue damage (see, Chapter 18 of Guides to the Evaluation of Permanent Impairment, Cocchiarella and Andersson, eds., Fifth Edition, 2001, American Medical Association Press).

The TNFα-defensin/chemokine fusion polypeptides and nucleotides encoding the TNFα-defensin/chemokine fusion polypeptides also find use in the treatment of TNFα-mediated pain resulting from a chronic inflammatory or autoimmune condition, for example, rheumatoid arthritis, Crohn's syndrome, inflammatory bowel disease.

The methods are carried out by administering a therapeutically effective amount of a TNFα-defensin/chemokine fusion polypeptide or a nucleotide encoding a TNFα-defensin/chemokine fusion polypeptide to a mammalian subject. The mammalian host usually is a human or a primate. In some embodiments, the mammalian host can be a domestic animal, for example, canine, feline, lagomorpha, rodentia, rattus, hamster, murine. In other embodiment, the mammalian host is an agricultural animal, for example, bovine, ovine, porcine, equine, etc.

In some embodiments, the TNFα-defensin/chemokine compositions are administered with an adjuvant, as described above.

a. Administration of Fusion Polypeptides

The fusion polypeptide of this invention can be administered to the subject orally or parenterally, and are usually administered parenterally, for example, intravenously, intradermally, subcutaneously, intramuscularly, transdermally. The fusion polypeptides are administered in an amount sufficient to induce an anti-TNFα immune response in the individual. Doses of the fusion polypeptide will vary from subject to subject, depending on the species, age, weight and general condition of the subject, the severity of the pain that is being treated, the particular fusion sequence being used, the mode of administration, and the like. Thus, it is not possible to specify an exact amount. However, an appropriate amount can be determined by one of ordinary skill in the art using only routine screening given the teachings herein. For example, to determine an appropriate dose of the fusion polypeptide to administer, pharmaceutically acceptable compositions comprising varying concentrations of the fusion polypeptide are prepared, administered to a subject and the immunological response to each dose is determined, for example, using well known ELISA techniques. See, Arnon, (ed.), Synthetic Vaccines, 1987, CRC Press, Boca Raton, Fla. and The Elisa Guidebook, Crowther (Editor), 2000, Humana Pr.

The TNFα fusion polypeptides are administered in an amount sufficient to elicit a neutralizing immune response directed against TNFα. Generally, the dosage of fusion protein will approximate that which is typical for the administration of vaccines, and typically, the dosage will be in the range of about 1 to 500 μg of the fusion polypeptide per dose, and preferably in the range of 50 to 250 μg of the fusion polypeptide per dose. This amount can be administered to the subject once every other week for about eight weeks or once every other month for about six months. The effects of the administration of the fusion polypeptide can be determined starting within the first month following the initial administration and continued thereafter at regular intervals, as needed, for an indefinite period of time.

b. Administration of Nucleic Acids Encoding Fusion Polypeptides

In one embodiment, nucleic acid sequences encoding the TNFα-defensin/chemokine fusion polypeptides can be delivered as naked DNA, optionally with adjuvant, as described above. Naked DNA vaccines and methods for the use of nucleic acids as DNA vaccines are generally known in the art; see, Wolff, et al., Science (1990) 247:1465; Brower, Nature Biotechnology (1998) 16:1304; Wolff, et al., Adv Genet (2005) 54:3; DNA Vaccines, Ertl, ed., 2003, Kluwer Academic Pub and DNA Vaccines: Methods and Protocols, Lowrie and Whalen, eds., 1999, Humana Press. The methods include placing a nucleic acid encoding the TNFα-defensin/chemokine fusion nucleotides under the control of a promoter for expression in a patient. Co-administering an adjuvant can further enhance the immune response against the TNFα-defensin/chemokine fusion polynucleotides. Without being bound by theory, following expression of the fusion polypeptide encoded by the DNA vaccine, cytotoxic T-cells, helper T-cells and antibodies are induced which recognize and destroy or eliminate cells or pathogens expressing the antigen.

Naked DNA can be delivered in solution (e.g., a phosphate-buffered saline solution) by injection, usually by an intra-arterial, intravenous, intranasal, subcutaneous or intramuscular route. The TNFα-defensin/chemokine fusion nucleotides are administered in an amount sufficient to elicit a neutralizing immune response directed against TNFα. In general, the dose of a naked nucleic acid composition is from about 10 μg to about 50 mg for a typical 70 kilogram patient. The actual dose will depend on several factors, including the species, age, weight and general condition of the subject, the severity of the pain that is being treated, the particular fusion sequence being used, the mode of administration, as discussed above. Subcutaneous or intramuscular doses for naked nucleic acid (typically DNA encoding a fusion protein) will range from 0.1 mg to 50 mg for a 70 kg patient in generally good health. This amount can be administered to the subject once every other week for about eight weeks or once every other month for about six months. The effects of the administration of the fusion nucleic acid sequences can be determined starting within the first month following the initial administration and continued thereafter at regular intervals, as needed, for an indefinite period of time.

In some embodiments, TNFα-defensin/chemokine fusion nucleic acid compositions are administered by liposome-based methods, electroporation or biolistic particle acceleration. A delivery apparatus (e.g., a “gene gun”) for delivering DNA into cells in vivo can be used. Such an apparatus is commercially available (e.g., BioRad, Hercules, Calif., or Chiron Vaccines, Emeryville, Calif.). Naked DNA can also be introduced into cells by complexing the DNA to a cation, such as polylysine, which is coupled to a ligand for a cell-surface receptor (see, for example, Wu, G. and Wu, C. H. (1988) J. Biol. Chem. 263:14621; Wilson et al. (1992) J. Biol. Chem. 267:963-967; and U.S. Pat. Nos. 5,166,320; 6,846,809; 6,733,777; 6,720,001; 6,290,987). Liposome formulations for delivery of naked DNA to mammalian host cells are commercially available from, for example, Encapsula NanoSciences, Nashville, Tenn. Other companies providing DNA vaccine delivery formulations include Lipoxen, London, England; Chiron Vaccines, and PowderMed, Oxford, England. An electroporation apparatus for use in delivery of naked DNA to mammalian host cells is commercially available from, for example, Inovio Biomedical Corporation, San Diego, Calif.

In one embodiment, the nucleic acid sequences encoding the TNFα-defensin/chemokine fusion polypeptides can be delivered in a viral vector, for example, an adenoviral or retroviral vector. Viral delivery systems are known in the art. In one embodiment, a retroviral vector system is used, which can package a recombinant retroviral genome (see, e.g., Young, et al., J Pathol. (2006) 208(2):299-318; and Anderson and Hope, Gene Ther. (2005) 12(23):1667-78). The recombinant retrovirus can then be used to infect and thereby deliver to the infected cells nucleic acid encoding the fusion polypeptide. The exact method of introducing the exogenous nucleic acid into mammalian cells is, of course, not limited to the use of retroviral vectors. Other techniques are widely available for this procedure including the use of adenoviral vectors or adeno-associated viral (AAV) vectors (see, e.g., Vaccine. (2006) 24(7):849-62; Wu, et al., Curr Gene Ther. (2005) 5(2):237-47; and Vanniasinkam, et al., Curr Gene Ther. (2005) 5(2):203-12), lentiviral vectors (see, e.g., Lever, et al., J Biomed Sci. (2004) 11(4):439-49 and Sinn, et al., Gene Ther. (2005) 12(14):1089-98), pseudotyped retroviral vectors (see, e.g., Verhoeyeri and Cosset, J Gene Med. (2004) 6 Suppl 1:S83-94).

The nucleic acid encoding the fusion protein can be inserted within a viral genome and the fusion protein encoding sequence can be positioned such that a virus promoter is operatively linked to the fusion protein nucleic acid insert such that the viral promoter can then direct transcription of the nucleic acid, or the fusion protein insert can contain its own viral promoter. Similarly, the fusion protein insert can be positioned such that the nucleic acid encoding the fusion protein can use other viral regulatory regions or sites such as splice junctions and polyadenylation signals and/or sites. Alternatively, the nucleic acid encoding the fusion protein can contain a different (i.e., heterologous) enhancer/promoter (e.g., CMV or RSV-LTR enhancer/promoter sequences) or other regulatory sequences, such as splice sites and polyadenylation sequences, such that the nucleic acid encoding the fusion protein can contain those sequences necessary for expression of the fusion protein and not partially or totally require these regulatory regions and/or sites of the virus genome. These regulatory sites can also be derived from another source, such as another virus. For example, a polyadenylation signal from SV40 or BGH can be used rather than a native viral, a human, or a murine polyadenylation signal. The fusion protein nucleic acid insert may, alternatively, contain some sequences necessary for expression of the nucleic acid encoding the fusion protein and derive other sequences necessary for the expression of the fusion protein nucleic acid from the virus genome, or even from the host in which the recombinant virus is introduced.

c. In Vitro Test of Immune Response

The generation of an anti-TNFα immune response can be determined using methods known in the art. In one embodiment, an anti-TNFα immune response is evaluated by measuring the concentration or titer of anti-TNFα antibodies in the serum of an individual that has been administered a TNFα-defensin/chemokine fusion polypeptide or fusion polynucleotide. This can be done, for example, using immunoassay techniques known in the art. For example, immunoassay plates, commercially available from, for example, Corning Life Sciences or Nunc, can be coated with a known amount of mammalian TNFα polypeptide, commercially available from, for example, R&D Systems, Minneapolis, Minn. or Peprotech, Rocky Hill, N.J. In some embodiments, the immunoassay plate is first coated with an anti-TNFα capture antibody. Unbound TNFα polypeptide is washed away, and then incubated with one or more dilutions of serum from the treated individual, for example, undiluted, 1:1, 1:10, 1:100, etc. Dilutions of serum can be, but need not be, serial.

Unbound proteins in the serum are washed away, and then bound anti-TNFα antibodies are incubated with a secondary antibody labeled with a detectable Moiety. The secondary antibody is specific for the immunoglobulin constant region of the species of treated individual, and can be labeled with an enzyme (e.g., horseradish peroxidase, alkaline phosphatase), a chemiluminescent moiety, a fluorescent moiety, a radioactive isotope, etc.

Unbound secondary antibody is washed away and bound secondary antibody is detected using an appropriate quantifying instrument, for example, a spectrophotometer, a fluorometer, a gamma-counter. Protocols for carrying out immunoassays are described in numerous sources, including for example, Current Protocols in Immunology, supra; The Elisa Guidebook, supra; and Harlow and Lane, Using Antibodies: A Laboratory Manual, 1998, Cold Spring Harbor Laboratory. The signals in the test reactions subject to serum are compared to a standard curve from control reactions containing known amounts of TNFα and known amounts of a characterized anti-TNFα antibody. The signals in test reactions are also compared to reactions containing serum from unimmunized individuals. A signal that is at least 20% greater than the signal from a reaction from an unimmunized subject is considered positive. Kits and reagents for developing an anti-TNFα immunoassay are commercially available from, for example, R&D Systems.

d. In Vivo Test of Ameliorating Chronic Pain Due to TNFα

The effectiveness of the TNFα-defensin/chemokine fusion polypeptides or fusion polynucleotides in eliciting an anti-TNFα immune response that ameliorates chronic neuropathic pain can be measured in vivo in rats or mice using a chronic constriction injury (CCI) model. This animal model of chronic neuropathic pain is described, for example, in Sommer and Schäfers, Brain Research (1998) 784:154-162; Sommer, et al., Exp Neurol (1998) 151:138-142; Sommer, et al., Brain Research (2001) 913:86-89; Vogel, et al., J Neurosci (2003) 23:708-715; and Covey, et al., Reg Anesth Pain Med (2002) 4:357-366.

Animals can be either immunized and then subjected to surgery or first subjected to surgery and then immunized. Usually, animals are first immunized with a TNFα-defensin/chemokine fusion polypeptide or fusion polynucleotide intraperitoneally, intradermally, subcutaneously, intramuscularly, intranasally or intravenously, at periodic intervals until anti-TNFα antibodies are detected in the serum. The sciatic nerve is then injured by constriction and the wound closed before behavioral testing begins. Control animals are not immunized and/or are not subjected to surgery, or are subjected to sham surgery that exposes but does not injure the sciatic nerve.

To determine responses to noxious (i.e., pain) stimuli, the animals are tested for thermal hyperalgesia, mechanical allodynia and cold allodynia. Baseline response values are determined in the 2-3 consecutive days before CCI surgery. Behavioral testing can begin on day 3 after surgery or after surgery and a vaccination regimen.

To determine thermal hyperalgesia, the plantar surface of a paw (e.g., a front paw or a hind paw) is subjected to radiant heat and the time for removal of the paw from the heat is measured (paw-withdrawal latency) (see, Hargreaves, et al., Pain (1988) 32:77). For each animal, the mean withdrawal latency time of several tests is measured, using alternating paws and resting between tests. A significant decrease in the mean withdrawal latency after CCI surgery compared with the baseline is defined as thermal hyperalgesia.

To determine mechanical allodynia, the plantar surface of a paw is probed with calibrated von Frey hairs that bend in response to a range of forces (e.g., 0.05-6.0 gm) and the 50% withdrawal threshold recorded (see, Chaplan, et al., J Neurosci Methods (1994) 53:55-63 and Sommer and Schäfers, Brain Res (1998)784:154). The 50% withdrawal threshold refers to the force of the von Frey hair to which an animal reacts in 50% of the presentations. For each animal, the mean 50% withdrawal threshold of several tests is measured. A significant decease in the 50% withdrawal threshold after CCI compared with the baseline is defined as mechanical allodynia.

To determine cold allodynia, a drop of acetone is contacted to the plantar aspect of a paw. The acetone can be delivered through a narrow-diameter tube (e.g., less than 1.0 mm) placed under the paw. A drop of water at 37° C. from a similar tube is delivered as a control. The paw elevation time is measured from the onset of the paw withdrawal until the paw is rested again for at least 2 seconds. For each animal, the mean withdrawal of several tests is measured. A response to acetone is defined as a sharp withdrawal of the paw lasting more than 1 second. A significant increase in postoperative paw withdrawal time to acetone compared with baseline is defined as cold allodynia.

Animals subjected to CCI but not immunized with a TNFα-defensin/chemokine fusion polypeptide or nucleotide will exhibit significantly increased responses to noxious stimuli in comparison to baseline response readings. Untreated animals, animals subjected to sham CCI and animal subjected to CCI and immunization with a TNFα-defensin/chemokine fusion sequence will not exhibit significantly increased responses to noxious stimuli in comparison to baseline response readings.

For statistical analysis, The Statistical Program for the Social Sciences (version 14.0; SPSS, Chicago, Ill.) or other appropriate software can be used. To compare behavioral data between groups and test days, a two-way repeated-measures ANOVA is used for parametric analysis, followed by a Student's t test. For nonparametric analysis of the von Frey thresholds, a Mann-Whitney U test is used for comparison of data between groups; a Friedman test is used for comparison of data between test days within a group, followed by a Wilcoxon test. A χ² test is used to compare groups at individual test days. A paired Student's t test is used to compare between two means in the same subject. Significance is assumed at p<0.05.

### Examples

The following examples are offered to illustrate, but not to limit the claimed invention.

### Example 1

### Murine TNFα Fusion Polypeptides

The following murine TNFα-defensin/chemokine fusion polypeptides are created either recombinantly or synthetically. The murine TNFα-defensin/chemokine fusion polypeptides comprise a mature murine TNFα polypeptide or immunogenic fragment thereof (e.g., SEQ ID NOs: 4, 36, 39, 41, 44 or 47) operably linked to a murine mature beta-defensin (e.g., SEQ ID NO:22) or a murine chemokine, either MCP-3 (e.g., SEQ ID NO:30) or IP-10 (e.g., SEQ ID NO:32). The fusion polypeptides optionally include a short flexible linker joining the TNFα sequence to the defensin or chemokine (e.g., a flexible Gly-Ser linker). The fusion polypeptides can be chemically synthesized by CSS, Chemical Synthesis Services, East Lothian, Scotland.

Alternatively, nucleotides encoding the murine TNFα-defensin/chemokine fusion polypeptides are chemically synthesized with overhangs corresponding to restriction sites in the multiple cloning region of a vector for expressing the fusion polypeptides in a desired host cell. The fusion nucleotides can be chemically synthesized by Integrated DNA Technologies of Coralville, Iowa. For expression in a bacterial host cell, e.g., E. coli, the synthetic fusion nucleotide can be cloned into a pET E. coli T7 expression vector or another appropriate vector from Novagen/EMD Biosciences, San Diego, Calif. For expression in a mammalian host cell, the synthetic fusion nucleotide can be cloned into a pcDNA vector (Invitrogen, Carlsbad, Calif.) or an appropriate mammalian expression vector from Promega, Madison, Wis.

### Example 2

### Immunizing Mice Against TNFα can Alleviate Neuropathic Pain Responses

The following example demonstrates eliciting an anti-TNFα immune response in an animal, and ameliorating chronic pain by vaccinating against TNFα using the TNFα-defensin/chemokine fusion sequences of the invention. The animals are divided into at least four groups: (1) immunized and subjected to constriction injury of the sciatic nerve; (2) immunized only; (3) immunized and subjected to constriction injury of the sciatic nerve; and (4) unimmunized and not subjected to nerve injury (e.g., sham surgery where the nerve is not injured).

Immunizing Mice with TNFα Fusion Sequence

C57B1/6 mice (or another wild-type strain, purchased from, for example, Jackson Labs, Bar Harbor, Me.) are inoculated intradermally with 5-20 μg of a fusion polypeptide or a fusion polynucleotide comprising a murine TNFα fusion polypeptide. Mice are inoculated intradermally three times at 2-week intervals. Sera are collected at each injection and 2 weeks after the final boost. When complete Freund's adjuvant (CFA) is used, the fusion polypeptide is diluted 1:1 in CFA for the initial injection and diluted 1:1 in incomplete. Freund's adjuvant (IFA) for subsequent inoculations. Vaccinations can also include an adjuvant, for example, Titermax Gold (CytRx Corp., Atlanta, Ga., USA) at 20% of the total volume, or CpG oligonucleotide 1826 (30 μg per injection) (Chu, et al., J Exp Med (1997) 186:1623).

ELISA Analysis of Antibody Induction.

Mouse antibodies to recombinant biologically active TNFα (PeproTech Inc., Rocky Hill, N.J., USA) is detected by ELISA using 200 ng of the target protein per well. Mouse serum is serially diluted in PBS-0.5% milk and applied to wells. Reactivity to these target proteins is determined using horseradish peroxidase-labeled goat anti-mouse IgG (or IgM) (Boehringer Mannheim Biochemicals Inc.) at a dilution of 1:2,000 as a secondary antibody. Upon development, optical densities (ODs) are read at 405 nm by a ThermoMax microplate reader. OD405 values that are greater than twice background (usually >0.1) are considered positive.

Chronic Constrictive Injury (CCI) Model of Neuropathic Pain

Under deep barbiturate anesthesia, a chronic constrictive injury (CCI) of one sciatic nerve is performed in the mice as described by Bennett and Xie, Pain (1988)33:87-107; Sommer and Schäfers, Brain Res (1998) 784:154-162 and Vogel, et al., J Neurosci (2003) 23:708-715. Briefly, three ligatures (10-0 Prolene or 7-0 Prolene) are placed around the sciatic nerve proximal to the trifurcation with 1 mm spacing, and tied until they just slightly constrict the diameter of the nerve and a brief twitch is seen in the respective hindlimb. This corresponds to blanching of the nerve and a significant reduction in nerve blood flow secondary to occlusion of the epineurial vasculature (Myers, et al., Anesthesiology (1993) 78:308-316). The wound is closed in layers.

Behavioral Testing

Testing of different sensory modalities is performed several consecutive days before CCI and at regular intervals thereafter and in controls. Only two tests are performed on the same day: (i) the measurement of skin temperature, (ii) the testing of heat sensitivity, (iii) the assessment of mechanical sensitivity, or (iv) the testing of cold sensitivity. The first day post-surgery serves to adapt the animals to the testing procedures. Mean values taken 2-3 days before surgery are taken as a mean to determine the baseline. Behavioral testing commences daily starting on day 3 following surgery. The experimenter is kept unaware of the animals' treatment group. Individual animals are excluded from the final analysis if the values of repetitive tests differ by more than 100%.

Thermal Hyperalgesia

Sensitivity to noxious heat is assessed according to the methods of Hargreaves, et al., Pain (1988) 32:77-88 and Lindenlaub, et al., Brain Res (2000) 866:15-22. Devices for carrying out thermal hypersensitivity testing are commercially available from Ugo Basile (Comerio, Italy). A radiant heat source is focused on the plantar surface of the hindpaw; the time from the initiation of the radiant heat until paw withdrawal is measured automatically [paw-withdrawal latency (PWL)]. A maximal cutoff of 20 sec is used to prevent tissue damage. Each paw is tested five times, alternating between paws with an interval of at least 1 min between tests; the mean withdrawal latency is calculated. The interval between two trials on the same paw is at least 5 min. A significant decrease in the mean withdrawal latency after CCI compared with the baseline is defined as heat hyperalgesia.

Mechanical Allodynia

Mechanical sensitivity is determined by probing the plantar surface of the hindpaw with calibrated von Frey hairs with circular plain tips of 0.8 mm diameter made from nylon filaments. The force required to bend the hairs ranged from 0.07 to 5.5 gm. Hairs are applied six times each on the basis of the up-and-down method of Dixon, et al., J Am Stat Assoc (1965) 60:967-978, according to Chaplan et al., J Neurosci Methods (1994) 53:55-63 and modified for mice (Sommer and Schäfers, Brain Res, (1998) 784:154-162). The time interval between two trials is at least 1 min on the same paw and at least 30 sec on the alternate paw.

The 50% withdrawal threshold (i.e., force of the von Frey hair to which an animal reacts in 50% of the presentations) is recorded. A significant decrease in the 50% withdrawal threshold after CCI compared with the baseline is termed mechanical allodynia (see, Sommer and Schäfers, supra).

Cold Allodynia

Cold stimulation of the animals' hindpaws is performed according to the method of Choi, et al., Pain (1994) 59:369-376, modified for mice (Vogel et al., Proceedings of the Ninth World Congress on Pain (2000) pp 249-257. Briefly, a drop of acetone is formed at the end of a polyethylene tube with a tip diameter of 0.8 mm connected with a syringe and gently applied at the plantar aspect of the hindpaw. A drop of water from a similar tube with a temperature of 37° C. serves as a control. A response to acetone is defined as sharp withdrawal of the hindpaw lasting less than 1 sec. Very brief withdrawals lasting less than 1 sec are assigned a value of 0 because they could be occasionally induced by a drop of water, whereas longer withdrawal times are typically a response to acetone and are generally not observed after the application of water. The paw elevation time is measured with a digital stopwatch from the onset of the paw withdrawal until the paw is rested again for at least 2 sec. The acetone is applied three times on each paw, and the mean of three trials is calculated. Repetitive testing is performed with an interval of at least 5 min for the same paw and of at least 1 min for the contralateral paw. Cold allodynia is defined as a significant increase in postoperative paw withdrawal time to acetone of individual animals compared with their baseline.

### Example 3

### Immunizing a Subject Against TNFα can Alleviate Pain

Patients with chronic pain secondary to pathological TNFα production, particularly neuropathic pain, are immunized one or more times with a fusion sequence of the invention. For example, subjects are administered one or more times, at about 3-4 week intervals, with up to about 500 μg of a fusion polypeptide (e.g., intradermally or subcutaneously) or up to about 500 μg of a nucleic acid encoding a fusion polypeptide (e.g., intradermally, subcutaneously, intranasally or intramuscularly). With each administration, the fusion polypeptide, the nucleic acid, or both are administered.

Patients receiving immunizations of the TNFα fusion sequences of the invention will have a statistically significant (i.e., p≦0.05 using an appropriate statistical test) reduction in serum TNFα concentrations, increase in anti-TNFα antibody concentrations in comparison to their pre-treatment status or in comparison to untreated patients.

Determination of the amelioration of chronic pain can be measured using any method known in the art. The measurement can be subjective or objective. For example, a patient can self report whether the pain is improved after receiving immunizations of the TNFα fusion sequences in comparison to their pre-treatment pain. Chronic pain can also be objectively measured, for example, by mapping radiofrequency (RF) emissions generated by the brain indicative of pain. This is described, for example, in U.S. Patent Publication No. 2006/0089551, hereby incorporated herein by reference. Generally, pain occurring in a patient's body sends pain impulses to the brain, where they are initially processed at the prefrontal area of the brain, including the cortical and subcortical portions thereof. Such pain impulses cause neuron activity in the prefrontal area of the brain, which can be observed on functional magnetic resonance images (“f-MRI”). Thus, the application of a pain stimulus, such as vibration, pressure or heat, to any part of the patient's body can be observed by increased neuron activity in the brain, at least initially principally in the prefrontal area thereof. However, the observable neuron activity in response to an applied pain stimulus differs between patients who suffer chronic pain and persons who are free of such pain. Specifically, the chronic pain sufferer is much more sensitive to the applied pain stimulus than a person free of chronic pain, which shows up on the f-MRI as increased neuron activity in the brain as compared to the f-MRI of a person free of chronic pain to whom the same pain stimulus was applied.

Patients receiving immunizations of the TNFα fusion sequences of the invention will have a statistically significant (i.e., p≦0.05 using an appropriate statistical test) reduction in pain, by objective or subjective measurements, in comparison to pretreatment or untreated patients. In particular, patients receiving immunizations of the TNFα fusion sequences will show statistically significant decreases in neuron activity in the brain in response to a pain stimulus as measured in an f-MRI in comparison to the patient's f-MRI in response to a pain stimulus before receiving the immunizations. The statistically significant decreases in neuron activity in the brain as measured in an f-MRI can manifest after one, two, three or more immunizations.

### Example 4

### Immunizing a Subject Against TNFα can Alleviate Insulin Resistance and/or Diabetes

Patients with hyperinsulinemia (serum insulin levels greater than 10 μIU/ml or greater than 69.5 pmol/L) or impaired glucose tolerance (fasting glucose levels of about 110-125 mg/dL or about 6.11-6.94 mmol/L) are immunized one or more times with a fusion sequence of the invention. For example, subjects are administered one or more times, at about 3-4 week intervals, with up to about 500 μg of a fusion polypeptide (e.g., intradermally or subcutaneously) or up to about 500 μg of a nucleic acid encoding a fusion polypeptide (e.g., intradermally, subcutaneously, intranasally or intramuscularly). With each administration, the fusion polypeptide, the nucleic acid, or both are administered.

Serum TNFα concentrations are measured before and after each immunization using any technique known in the art. For example, TNFα levels can be measured using a standard immunoassay, commercially available from, for example, R&D Systems, Minneapolis, Minn. Anti-TNFα antibody titers are also measured, for example, using standard immunoassay methods. Measurements of serum insulin, glucose, and insulin resistance/sensitivity are also made before and after each immunization. Typically, measurements of serum insulin, glucose, and insulin resistance/sensitivity are made after about 12 hours of fasting. Insulin is measured by any technique known in the art, for example, by immunoassay, where kits are commercially available from, for example, Diagnostic Products Corp., Los Angeles, Calif. Insulin sensitivity/resistance can be measured using any methodology known in the art. For example, insulin sensitivity can be measured using the hyperinsulinemic/euglycemic clamp technique, by measuring fasting plasma insulin levels, intravenous glucose tolerance, homeostasis model assessment (HOMA), or quantitative insulin sensitivity check index (QUICKI). Insulin sensitivity/resistance can be quantitatively calculated using the Minimal Model, Los Angeles, Calif. See, for example, Bernstein, et al., Arch Intern Med (2006) 166:902; Arslanian, Horm Res (2006) 64 Suppl 3:16-24; Soop, et al., Clin Sci (Lond) (2000) 98:367-74; Straczkowski, et al., Med Sci Monit (2004) 10(8):CR480-4; Keijzers, et al., Diabetes Care (2002) 25:364-9; Tack, et al., Diabetologia (1998) 41:569-76; and Boston, et al., Diabetes Technol Ther. (2003) 5(6):1003-15. Blood glucose is measured using any method known in the art, for example, using a glucose oxidase method. See, Ofei, et al., Diabetes (1996) 45:881-885.

Patients receiving immunizations of the TNFα fusion sequences of the invention will have a statistically significant (i.e., p≦0.05 using an appropriate statistical test) reduction in serum TNFα concentrations, increase in anti-TNFα antibody concentrations, and reduction in insulin sensitivity in comparison to their pre-treatment status or in comparison to untreated patients. The statistically significant decreases in insulin sensitivity can manifest after one, two, three or more immunizations.

Serum levels of other metabolic markers including inflammation indicators (e.g., C-reactive protein (CRP), free fatty acids (FFA), interleukin-6 (IL-6), adiponectin, and/or fibrinogen, and cardiovascular indicators (e.g., high density lipoprotein (HDL), low density lipoprotein (LDL), total cholesterol, and/or triglycerides) also can be measured before and after each immunization using methodologies known in the art. For example, radio- and enzyme-linked-immunoassays are commercially available to measure levels of CRP (Diagnostic Systems Laboratory, Webster, Tex.), adiponectin (LINCO Research, Inc., St Charles, Mo.) and IL-6 (R&D Systems, Minneapolis, Minn.). Fibrinogen levels can be determined using an immunoturbidimetric assay (Kamiya Biomedical, Seattle, Wash.).

Nonesterified fatty acid concentrations can be measured using an in vitro enzymatic colorimetric assay kit (Wako Chemicals, Richmond, Va.). See, Bernstein, et al., Arch Intern Med (2006) 166:902.

Patients receiving immunizations of the TNFα fusion sequences of the invention will have a statistically significant (i.e., p≦0.05 using an appropriate statistical test) reduction in serum CRP, FFA, IL-6, adiponectin, fibrinogen, triglycerides, total cholesterol and/or LDL concentrations, and increase in HDL concentrations, in comparison to pretreatment or untreated patients.

### Example 5

### Immunizing a Subject Against TNFα can Alleviate Cardiovascular Disease

Patients with a serum triglyceride level of 150 mg/dL or higher (1.70 mmol/L) or a high density lipoprotein cholesterol level less than 35 mg/dL (0.91 mmol/L) for men or less than 39 mg/dL (1.01 mmol/L) for women or a total cholesterol level of greater than 6.0 mmol/L are immunized one or more times with a fusion sequence of the invention. For example, subjects are administered one or more times, at about 3-4 week intervals, with up to about 500 μg of a fusion polypeptide (e.g., intradermally or subcutaneously) or up to about 500 μg of a nucleic acid encoding a fusion polypeptide (e.g., intradermally, subcutaneously, intranasally or intramuscularly). With each administration, the fusion polypeptide, the nucleic acid, or both are administered.

Serum levels of indicators of cardiovascular disease, including total cholesterol, LDL, HDL, triglycerides and/or apolipoprotein AI and B are measured using any method known in the art. See, for example, Vis, et al., J Rheumatol (2005) 32:252-255; Popa, et al., Ann Rheum Dis (2005) 64:303-305; Irace, et al., Atherosclerosis (2004) 177:113-118. See also, information on diagnostic tests provided through the website for Quest Diagnostics, on the worldwide web at questdiagnostics.com.

Patients receiving immunizations of the TNFα fusion sequences of the invention will have a statistically significant (i.e., p≦0.05 using an appropriate statistical test) reduction in serum triglycerides, total cholesterol and/or LDL concentrations, and/or an increase in HDL concentrations, in comparison to pretreatment or untreated patients. The statistically significant decreases in serum triglycerides, total cholesterol and/or LDL concentrations, and/or increase in HDL concentrations can manifest after one, two, three or more immunizations.

Patients receiving immunizations of the TNFα fusion sequences of the invention will have a statistically significant (i.e., p≦0.05 using an appropriate statistical test) reduction in serum TNFα concentrations and increase in anti-TNFα antibody concentrations in comparison to their pre-treatment status or in comparison to untreated patients.

### Example 6

### Immunizing a Subject Against TNFα can Alleviate Chronic Inflammation

Patients suffering from a chronic inflammatory condition are immunized one or more times with a fusion sequence of the invention. For example, subjects are administered one or more times, at about 3-4 week intervals, with up to about 500 μg of a fusion polypeptide (e.g., intradermally or subcutaneously) or up to about 500 μg of a nucleic acid encoding a fusion polypeptide (e.g., intradermally, subcutaneously, intranasally or intramuscularly). With each administration, the fusion polypeptide, the nucleic acid, or both are administered.

Serum levels of inflammation markers including C-reactive protein (CRP), interleukin-6 adiponectin, and/or fibrinogen are measured before and after each immunization using methodologies known in the art. As discussed above, radio- and enzyme-linked-immunoassays are commercially available to measure levels of CRP (Diagnostic Systems Laboratory, Webster, Tex.), adiponectin (LINCO Research, Inc., St Charles, Mo.) and IL-6 (R&D Systems, Minneapolis, Minn.). Fibrinogen levels can be determined using an immunoturbidimetric assay (Kamiya Biomedical, Seattle, Wash.). Nonesterified fatty acid concentrations can be measured using an in vitro enzymatic colorimetric assay kit (Wako Chemicals, Richmond, Va.). See, Bernstein, et al., Arch Intern Med (2006) 166:902.

Patients receiving immunizations of the TNFα fusion sequences of the invention will have a statistically significant (i.e., p≦0.05 using an appropriate statistical test) reduction in serum CRP, IL-6, adiponectin and/or fibrinogen in comparison to pretreatment or untreated patients. The statistically significant decreases in serum CRP, IL-6, adiponectin and/or fibrinogen concentrations can manifest after one, two, three or more immunizations.

Patients receiving immunizations of the TNFα fusion sequences of the invention will have a statistically significant (i.e., p≦0.05 using an appropriate statistical test) reduction in serum TNFα concentrations and increase in anti-TNFα antibody concentrations in comparison to their pre-treatment status or in comparison to untreated patients.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A fusion polypeptide comprising a chemokine polypeptide or a defensin polypeptide operably linked to a TNFα polypeptide.

2. The fusion polypeptide of claim 1, wherein the chemokine is selected from the group consisting of interferon-induced protein 10 (IP-10), monocyte chemotactic protein-1 (MCP-1), MCP-2, MCP-3, MCP-4, macrophage inflammatory protein 1 (MIP1), MIP2, MIP3, RANTES (CC chemokine ligand 5), macrophage-derived chemokine (MDC), stromal cell-derived factor 1 (SDF-1), and monokine induced by IFN-gamma (MIG).

3. The fusion polypeptide of claim 1, wherein the defensin is an alpha defensin or a beta defensin.

4. The fusion polypeptide of claim 3, wherein the defensin is a beta defensin selected from the group consisting of HBD1 and HBD2, or an alpha defensin selected from the group consisting of HNP-1, HNP-2, and HNP-3.

5. The fusion polypeptide of claim 1, wherein the polypeptide has an amino acid sequence having at least 95% sequence identity to a fusion protein comprising a first polypeptide segment selected from the group consisting of SEQ ID NO:8, SEQ ID NO:10, SEQ ID NO:12, SEQ ID NO:14, SEQ ID NO:16, SEQ ID NO:18, SEQ ID NO:24 and SEQ ID NO:26 operably linked to a second polypeptide segment selected from the group consisting of SEQ ID NO:2, SEQ ID NO:35, SEQ ID NO:38 and SEQ ID NO:40.

6. The fusion polypeptide of claim 1, further comprising a pharmaceutically acceptable carrier.

7. A nucleic acid encoding a fusion polypeptide comprising a TNFα polypeptide fused to a chemokine polypeptide or a defensin polypeptide.

8. The nucleic acid of claim 7, wherein the nucleic acid has a nucleotide sequence having at least 95% sequence identity a nucleotide encoding a fusion protein, the nucleotide sequence comprising a first nucleotide segment selected from the group consisting of SEQ ID NO:7, SEQ ID NO:9, SEQ ID NO:11, SEQ ID NO:13, SEQ ID NO:15, SEQ ID NO:17, SEQ ID NO:23 and SEQ ID NO:25 operably linked to a second nucleotide segment comprising SEQ IL) NO:1 or a fragment thereof encoding an immunogenic polypeptide.

9. The nucleic acid of claim 7, further comprising a pharmaceutically acceptable carrier.

10. A vector comprising the nucleic acid of claim 7.

11. A cell comprising the vector of claim 10.

12. A method of producing an immune response in a subject specifically directed against TNFα, comprising administering to the subject the fusion polypeptide of claim 6.

13. A method of producing an immune response in a subject specifically directed against TNFα, comprising administering to the subject the nucleic acid of claim 9.

14. A method of inhibiting or preventing chronic pain mediated by TNFα in a subject comprising, administering to the subject the fusion polypeptide of claim 6.

15. The method of claim 14, wherein the chronic pain is neuropathic pain.

16. The method of claim 14, wherein the chronic pain is chronic back pain.

17. A method of inhibiting or preventing chronic pain mediated by TNFα in a subject comprising, administering to the subject the nucleic acid of claim 9.

18. A method of inhibiting or preventing a disease condition mediated by pathological TNFα in a subject comprising, administering to the subject the fusion polypeptide of claim 6 or the nucleic acid of claim 9.

19. The method of claim 18, wherein the disease condition is selected from the group consisting of chronic inflammation, chronic neuropathic pain, diabetes and cardiovascular disease.


SEQUENCE LISTING (68 sequences)

SEQ ID NO: 1 - DNA, 471 bp, Homo sapiens
  human tumor necrosis factor-alpha (hTNFalpha) mature form
gtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctg

SEQ ID NO: 2 - protein, 157 aa, Homo sapiens [sequence omitted]

SEQ ID NO: 3 - DNA, 468 bp, Mus musculus
  mouse tumor necrosis factor-alpha (mTNFalpha) mature form
ctcagatcatcttctcaaaattcgagtgacaagcctgtagcccacgtcgtagcaaaccaccaagtggaggagcagctggagtggctgagccagcgcgccaacgccctcctggccaacggcatggatctcaaagacaaccaactagtggtgccagccgatgggttgtaccttgtctactcccaggttctcttcaagggacaaggctgccccgactacgtgctcctcacccacaccgtcagccgatttgctatctcataccaggagaaagtcaacctcctctctgccgtcaagagcccctgccccaaggacacccctgagggggctgagctcaaaccctggtatgagcccatatacctgggaggagtcttccagctggagaagggggaccaactcagcgctgaggtcaatctgcccaagtacttagactttgcggagtccgggcaggtctactttggagtcattgctctg

SEQ ID NO: 4 - protein, 156 aa, Mus musculus
  mouse tumor necrosis factor-alpha (mTNFalpha) mature form
LRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 5 - DNA, 468 bp, Rattus norvegicus
  rat tumor necrosis factor-alpha (rTNFalpha) mature form
ctcagatcatcttctcaaaactcgagtgacaagcccgtagcccacgtcgtagcaaaccaccaagcagaggagcagctggagtggctgagccagcgtgccaacgccctcctggccaatggcatggatctcaaagacaaccaactggtggtaccagcagatgggctgtaccttatctactcccaggttctcttcaagggacaaggctgccccgactatgtgctcctcacccacaccgtcagccgatttgccacttcataccaggagaaagtcagcctcctctccgccatcaagagcccttgccctaaggacacccctgagggagctgagctcaagccctggtatgagcccatgtacctgggaggagtctcccagctggagaagggggacctgctcagcgctgaggtcaacctgcccaagtacttagacatcacggagtccgggcaggtctactttggagtcattgctctg

SEQ ID NO: 6 - protein, 156 aa, Rattus norvegicus
  rat tumor necrosis factor-alpha (rTNFalpha) mature form
LRSSSQNSSDKPVAHVVANHQAEEQLEWLSQRANALLANGMDLKDNQLVVPADGLYLIYSQVLFKGQGCPDYVLLTHTVSRFATSYQEKVSLLSAIKSPCPKDTPEGAELKPWYEPMYLGGVSQLEKGDLLSAEVNLPKYLDITESGQVYFGVIAL

SEQ ID NO: 7 - DNA, 90 bp, Homo sapiens
  human defensin-alpha-1 (human neutrophil peptide-1, HNP-1) mature form
gcctgctattgcagaataccagcgtgcattgcaggagaacgtcgctatggaacctgcatctaccagggaagactctgggcattctgctgc

SEQ ID NO: 8 - protein, 30 aa, Homo sapiens
  human defensin-alpha-1 (human neutrophil peptide-1, HNP-1) mature form
ACYCRIPACIAGERRYGTCIYQGRLWAFCC

SEQ ID NO: 9 - DNA, 87 bp, Homo sapiens
  human defensin-alpha-2 (human neutrophil peptide-2, HNP-2) mature form
tgctattgcagaataccagcgtgcattgcaggagaacgtcgctatggaacctgcatctaccagggaagactctgggcattctgctgc

SEQ ID NO: 10 - protein, 29 aa, Homo sapiens
  human defensin-alpha-2 (human neutrophil peptide-2, HNP-2) mature form
CYCRIPACIAGERRYGTCIYQGRLWAFCC

SEQ ID NO: 11 - DNA, 90 bp, Homo sapiens
  human defensin-alpha-3 (human neutrophil peptide-3, HNP-3) mature form
gactgctattgcagaataccagcgtgcattgcaggagaacgtcgctatggaacctgcatctaccagggaagactctgggcattctgctgc

SEQ ID NO: 12 - protein, 30 aa, Homo sapiens
  human defensin-alpha-3 (human neutrophil peptide-3, HNP-3) mature form
DCYCRIPACIAGERRYGTCIYQGRLWAFCC

SEQ ID NO: 13 - DNA, 110 bp, Homo sapiens
  human defensin-alpha-4 (human neutrophil peptide-4, HNP-4) mature form
gtctgctcttgcagattagtattctgccggcgaacagaacttcgtgttgggaactgcctcattggtggtgtgagtttcacatactgctgcacgcgtgtcgattaacgttc

SEQ ID NO: 14 - protein, 34 aa, Homo sapiens
  human defensin-alpha-4 (human neutrophil peptide-4, HNP-4) mature form
VCSCRLVFCRRTELRVGNCLIGGVSFTYCCTRVD

SEQ ID NO: 15 - DNA, 108 bp, Homo sapiens
  human defensin-beta-1 (hBD-1) mature form
gatcattacaattgcgtcagcagtggagggcaatgtctctattctgcctgcccgatctttaccaaaattcaaggcacctgttacagagggaaggccaagtgctgcaag

SEQ ID NO: 16 - protein, 36 aa, Homo sapiens
  human defensin-beta-1 (hBD-1) mature form
DHYNCVSSGGQCLYSACPIFTKIQGTCYRGKAKCCK

SEQ ID NO: 17 - DNA, 114 bp, Homo sapiens
  human defensin-beta-2 (hBD-2) mature form
gatcctgttacctgccttaagagtggagccatatgtcatccagtcttttgccctagaaggtataaacaaattggcacctgtggtctccctggaacaaaatgctgcaaaaagcca

SEQ ID NO: 18 - protein, 38 aa, Homo sapiens
  human defensin-beta-2 (hBD-2) mature form
DPVTCLKSGAICHPVFCPRRYKQIGTCGLPGTKCCKKP

SEQ ID NO: 19 - DNA, 117 bp, Rattus norvegicus
  rat defensin-beta-2 (rBD-2) mature form
gattatcatcgatgtctaagagagaaagggttctgtctcaatgctgtttgtcctcgttctaccttgtttgtgggtacctgtttcccatacaagttctactgttgcaagtttaaaaga

SEQ ID NO: 20 - protein, 39 aa, Rattus norvegicus
  rat defensin-beta-2 (rBD-2) mature form
DYHRCLREKGFCLNAVCPRSTLFVGTCFPYKFYCCKFKR

SEQ ID NO: 21 - DNA, 117 bp, Mus musculus
  mouse defensin-beta-2 (mBD-2) mature form
gaacttgaccactgccacaccaatggagggtactgtgtcagagccatttgtcctccttctgccaggcgtcctgggagctgtttcccagagaagaacccctgttgcaagtacatgaaa

SEQ ID NO: 22 - protein, 39 aa, Mus musculus
  mouse defensin-beta-2 (mBD-2) mature form
ELDHCHTNGGYCVRAICPPSARRPGSCFPEKNPCCKYMK

SEQ ID NO: 23 - DNA, 230 bp, Homo sapiens
  human monocyte chemotactic protein-1 (MCP-1, CCL2)
ctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagact

SEQ ID NO: 24 - protein, 76 aa, Homo sapiens
  human monocyte chemotactic protein-1 (MCP-1, CCL2)
QPDAINAPVTCCYNFTNRKISVQRLASYRRITSSKCPKEAVIFKTIVAKEICADPKQKWVQDSMDHLDKQTQTPKT

SEQ ID NO: 25 - DNA, 297 bp, Homo sapiens
  human monocyte chemotactic protein-3 (MCP-3, CCL7)
atgaaagcctctgcagcacttctgtgtctgctgctcacagcagctgctttcagcccccaggggcttgctcagccagttgggattaatacttcaactacctgctgctacagatttatcaataagaaaatccctaagcagaggctggagagctacagaaggaccaccagtagccactgtccccgggaagctgtaatcttcaagaccaaactggacaaggagatctgtgctgaccccacacagaagtgggtccaggactttatgaagcacctggacaagaaaacccaaactccaaagctt

SEQ ID NO: 26 - protein, 99 aa, Homo sapiens
  human monocyte chemotactic protein-3 (MCP-3, CCL7)
MKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 27 - DNA, 291 bp, Rattus norvegicus
  rat monocyte chemotactic protein-3 (MCP-3, CCL7)
atgcagatctctgccgcgcttctgtgtgtgctgctcacagctgctgctttcaccgtgcacgtgtgggcccaaccagatgggaccaattcatccacttgctgctatgtcaagaaacaaaagattcccaagaggaatctcaagagttacagaaagatcaccagtagtcggtgtccctgggaagctgttatcttcaagaccaaaaagggcatggaagtctgtgctgaagcccatcagaagtgggttgaggaggccatagcatacttagacatgaaaacttcaactccaaagccc

SEQ ID NO: 28 - protein, 97 aa, Rattus norvegicus
  rat monocyte chemotactic protein-3 (MCP-3, CCL7)
MQISAALLCVLLTAAAFTVHVWAQPDGTNSSTCCYVKKQKIPKRNLKSYRKITSSRCPWEAVIFKTKKGMEVCAEAHQKWVEEAIAYLDMKTSTPKP

SEQ ID NO: 29 - DNA, 291 bp, Mus musculus
  mouse monocyte chemotactic protein-3 (MCP-3, CCL7)
atgaggatctctgccacgcttctgtgcctgctgctcatagccgctgctttcagcatccaagtgtgggcccaaccagatgggcccaatgcatccacatgctgctatgtcaagaaacaaaagatccccaagaggaatctcaagagctacagaaggatcaccagtagtcggtgtccctgggaagctgttatcttcaagacaaagaagggcatggaagtctgtgctgaagcccatcagaagtgggtcgaggaggctatagcatacttagacatgaaaaccccaactccaaagcct

SEQ ID NO: 30 - protein, 97 aa, Mus musculus
MRISATLLCLLLIAAAFSIQVWAQPDGPNASTCCYVKKQKIPKRNLKSYRRITSSRCPWEAVIFKTKKGMEVCAEAHQKWVEEAIAYLDMKTPTPKP

SEQ ID NO: 31 - DNA, 231 bp, Homo sapiens
  human interferon-induced protein 10 (IP-10)
gtacctctctctagaaccgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaatgtctaaaagatctcct

SEQ ID NO: 32 - protein, 77 aa, Homo sapiens
  human interferon-induced protein 10 (IP-10)
VPLSRTVRCTCISISNQPVNPRSLEKLEIIPASQFCPRVEIIATMKKKGEKRCLNPESKAIKNLLKAVSKEMSKRSP

SEQ ID NO: 33 - DNA, 294 bp, Mus musculus
  mouse interferon-induced protein 10 (IP-10)
atgaacccaagtgctgccgtcattttctgcctcatcctgctgggtctgagtgggactcaagggatccctctcgcaaggacggtccgctgcaactgcatccatatcgatgacgggccagtgagaatgagggccatagggaagcttgaaatcatccctgcgagcctatcctgcccacgtgttgagatcattgccacgatgaaaaagaatgatgagcagagatgtctgaatccggaatctaagaccatcaagaatttaatgaaagcgtttagccaaaaaaggtctaaaagggctcct

SEQ ID NO: 34 - protein, 98 aa, Mus musculus
  mouse interferon-induced protein 10 (IP-10)
MNPSAAVIFCLILLGLSGTQGIPLARTVRCNCIHIDDGPVRMRAIGKLEIIPASLSCPRVEIIATMKKNDEQRCLNPESKTIKNLMKAFSQKRSKRAP

SEQ ID NO: 35 - protein, 23 aa, Artificial Sequence
  Description of Artificial Sequencesynthetic aa 1-23 of human tumor necrosis factor-alpha (TNF-alpha) mature form
LRSSSQNSSDKPVAHVVANHQVE

SEQ ID NO: 36 - protein, 23 aa, Artificial Sequence
  Description of Artificial Sequencesynthetic aa 1-23 of mouse tumor necrosis factor-alpha (TNF-alpha) mature form
LRSSSQNSSDKPVAHVVANHQVE

SEQ ID NO: 37 - protein, 23 aa, Artificial Sequence
  Description of Artificial Sequencesynthetic aa 1-23 of rat tumor necrosis factor-alpha (TNF-alpha) mature form
LRSSSQNSSDKPVAHVVANHQAE

SEQ ID NO: 38 - protein, 19 aa, Artificial Sequence
  Description of Artificial Sequencesynthetic aa 37-55 of human tumor necrosis factor-alpha (TNF-alpha) mature form
LANGVELRDNQLVVPSEGL

SEQ ID NO: 39 - protein, 19 aa, Artificial Sequence
  Description of Artificial Sequencesynthetic aa 37-55 of mouse and rat tumor necrosis factor-alpha (TNF-alpha) mature form
LANGMDLKDNQLVVPADGL

SEQ ID NO: 40 - protein, 20 aa, Artificial Sequence
  Description of Artificial Sequencesynthetic aa 117-136 of human tumor necrosis factor-alpha (TNF-alpha) mature form
PIYLGGVFQLEKGDRLSAEI

SEQ ID NO: 41 - protein, 20 aa, Artificial Sequence
  Description of Artificial Sequencesynthetic aa 116-135 of mouse tumor necrosis factor-alpha (TNF-alpha) mature form
PIYLGGVFQLEKGDQLSAEV

SEQ ID NO: 42 - protein, 20 aa, Artificial Sequence
  Description of Artificial Sequencesynthetic aa 116-135 of rat tumor necrosis factor-alpha (TNF-alpha) mature form
PMYLGGVSQLEKGDLLSAEV

SEQ ID NO: 43 - protein, 22 aa, Artificial Sequence
  Description of Artificial Sequencesynthetic aa 95-116 of human tumor necrosis factor-alpha (TNF-alpha) mature form
SAIKSPCQRETPEGAEAKPWYE

SEQ ID NO: 44 - protein, 22 aa, Artificial Sequence
  Description of Artificial Sequencesynthetic aa 94-115 of mouse tumor necrosis factor-alpha (TNF-alpha) mature form
SAVKSPCPKDTPEGAELKPWYE

SEQ ID NO: 45 - protein, 22 aa, Artificial Sequence
  Description of Artificial Sequencesynthetic aa 94-115 of rat tumor necrosis factor-alpha (TNF-alpha) mature form
SAIKSPCPKDTPEGAELKPWYE

SEQ ID NO: 46 - protein, 21 aa, Artificial Sequence
  Description of Artificial Sequencesynthetic aa 137-157 of human tumor necrosis factor-alpha (TNF-alpha) mature form
NRPDYLDFAESGQVYFGIIAL

SEQ ID NO: 47 - protein, 21 aa, Artificial Sequence
  Description of Artificial Sequencesynthetic aa 136-156 of mouse tumor necrosis factor-alpha (TNF-alpha) mature form
NLPKYLDFAESGQVYFGVIAL

SEQ ID NO: 48 - protein, 21 aa, Artificial Sequence
  Description of Artificial Sequencesynthetic aa 136-156 of rat tumor necrosis factor-alpha (TNF-alpha) mature form
NLPKYLDITESGQVYFGVIAL

SEQ ID NO: 49 - DNA, 69 bp, Artificial Sequence
  Description of Artificial Sequencesynthetic nucleic acid encoding aa 1-23 of human tumor necrosis factor-alpha (TNF-alpha) mature form
gtcagatcatcttctcgaaccccgagtgacaagcctgtagcccatgttgtagcaaaccctcaagctgag

SEQ ID NO: 50 - DNA, 69 bp, Artificial Sequence
  Description of Artificial Sequencesynthetic nucleic acid encoding aa 1-23 of mouse tumor necrosis factor-alpha (TNF-alpha) mature form
ctcagatcatcttctcaaaattcgagtgacaagcctgtagcccacgtcgtagcaaaccaccaagtggag

SEQ ID NO: 51 - DNA, 69 bp, Artificial Sequence
  Description of Artificial Sequencesynthetic nucleic acid encoding aa 1-23 of rat tumor necrosis factor-alpha (TNF-alpha) mature form
ctcagatcatcttctcaaaactcgagtgacaagcccgtagcccacgtcgtagcaaaccaccaagcagag

SEQ ID NO: 52 - DNA, 57 bp, Artificial Sequence
  Description of Artificial Sequencesynthetic nucleic acid encoding aa 37-55 of human tumor necrosis factor-alpha (TNF-alpha) mature form
ctggccaatggcgtggagctgagagataaccagctggtggtgccatcagagggcctg

SEQ ID NO: 53 - DNA, 57 bp, Artificial Sequence
  Description of Artificial Sequencesynthetic nucleic acid encoding aa 37-55 of mouse and rat tumor necrosis factor-alpha (TNF-alpha) mature form
ctggccaacggcatggatctcaaagacaaccaactagtggtgccagccgatgggttg

SEQ ID NO: 54 - DNA, 66 bp, Artificial Sequence
  Description of Artificial Sequencesynthetic nucleic acid encoding aa 117-136 of human tumor necrosis factor-alpha (TNF-alpha) mature form
tctgccatcaagagcccctgccagagggagaccccagagggggctgaggccaagccctggtatgag

SEQ ID NO: 55 - DNA, 66 bp, Artificial Sequence
  Description of Artificial Sequencesynthetic nucleic acid encoding aa 116-135 of mouse tumor necrosis factor-alpha (TNF-alpha) mature form
tctgccgtcaagagcccctgccccaaggacacccctgagggggctgagctcaaaccctggtatgag

SEQ ID NO: 56 - DNA, 66 bp, Artificial Sequence
  Description of Artificial Sequencesynthetic nucleic acid encoding aa 116-135 of rat tumor necrosis factor-alpha (TNF-alpha) mature form
tccgccatcaagagcccttgccctaaggacacccctgagggagctgagctcaagccctggtatgag

SEQ ID NO: 57 - DNA, 60 bp, Artificial Sequence
  Description of Artificial Sequencesynthetic aa 95-116 of human tumor necrosis factor-alpha mature form
cccatctatctgggaggggtcttccagctggagaagggtgaccgactcagcgctgagatc

SEQ ID NO: 58 - DNA, 60 bp, Artificial Sequence
  Description of Artificial Sequencesynthetic aa 94-115 of mouse tumor necrosis factor-alpha mature form
cccatatacctgggaggagtcttccagctggagaagggggaccaactcagcgctgaggtc

SEQ ID NO: 59 - DNA, 60 bp, Artificial Sequence
  Description of Artificial Sequencesynthetic nucleic acid encoding aa 94-115 of rat tumor necrosis factor-alpha (TNF-alpha) mature form
cccatgtacctgggaggagtctcccagctggagaagggggacctgctcagcgctgaggtc

SEQ ID NO: 60 - DNA, 63 bp, Artificial Sequence
  Description of Artificial Sequencesynthetic nucleic acid encoding aa 137-157 of human tumor necrosis factor-alpha (TNF-alpha) mature form
aatcggcccgactatctcgactttgccgagtctgggcaggtctactttgggatcattgccctg

SEQ ID NO: 61 - DNA, 63 bp, Artificial Sequence
  Description of Artificial Sequencesynthetic aa 136-156 of mouse tumor necrosis factor-alpha (TNF-alpha) mature form
aatctgcccaagtacttagactttgcggagtccgggcaggtctactttggagtcattgctctg

SEQ ID NO: 62 - DNA, 63 bp, Artificial Sequence
  Description of Artificial Sequencesynthetic aa 136-156 of rat tumor necrosis factor-alpha (TNF-alpha) mature form
aacctgcccaagtacttagacatcacggagtccgggcaggtctactttggagtcattgctctg

SEQ ID NO: 63 - protein, 30 aa, Artificial Sequence
  Description of Artificial Sequencesynthetic alpha defensin consensus sequence
XCXCXXXXCXXXXXXXXXCXXXXXXXXXCC

SEQ ID NO: 64 - protein, 29 aa, Artificial Sequence
  Description of Artificial Sequencesynthetic consensus sequence of human neutrophil peptide-1 (HNP-1), human neutrophil peptide-2 (HNP-2) and human neutrophil peptide-3 (HNP-3)
CYCRIPACIAGERRYGTCIYQGRLWAFCC

SEQ ID NO: 65 - protein, 35 aa, Artificial Sequence
  Description of Artificial Sequencesynthetic beta defensin consensus sequence
XXXXCXXXXXXCXXXXCXXXXXXXXXCXXXXXXCC

SEQ ID NO: 66 - protein, 5 aa, Artificial Sequence
  Description of Artificial Sequencesynthetic fusion polypeptide intervening flexible linker
GGGGS

SEQ ID NO: 67 - protein, 25 aa, Artificial Sequence
  Description of Artificial Sequencesynthetic fusion polypeptide intervening flexible linker
GGGGSXXXXXXXXXXXXXXXXXXXX

SEQ ID NO: 68 - protein, 6 aa, Artificial Sequence
  Description of Artificial Sequencesynthetic 6 sequential histidines, HisTag, purification tag
HHHHHH